(19) United States
(12) Patent Application Publication
(10) Pub. No.: US 2024/0181168 A1
(43) Pub. Date: Jun. 6, 2024

(54) DEVICES AND SYSTEMS FOR DELIVERING THERAPEUTIC AGENTS

(71) Applicants: Deepak JAIN, Winston-Salem, NC (US); Timothy A. BERTRAM, George Town (KY)

(72) Inventors: Deepak JAIN, Winston-Salem, NC (US); Timothy A. BERTRAM, George Town (KY)

(21) Appl. No.: 17/996,571

(22) PCT Filed: Apr. 21, 2021
(86) PCT No.: PCT/US21/28342

§ 371 Date: Oct. 19, 2022

Related U.S. Application Data
(60) Provisional application No. 63/013,082, filed on Apr. 21, 2020.

(51) Int. Cl. A61M 5/32; A61B 17/34; A61M 5/142

(52) U.S. Cl. CPC A61M 5/32; A61B 17/3415; A61M 5/142; A61B 2017/348; A61B 17/3498

(57) ABSTRACT

Provided herein are, inter alia, devices, methods, and systems are provided for delivering pharmaceutical fluid formulations, such as with metered infusion capabilities delivered extravascularly to target locations within a patient while maintaining positional stability. An exemplary device can be configured to deliver non-continuous streams or boluses of fluid to a target site. In certain embodiments, the injection device can engage with an exemplary trocar that is configured to have one or more stabilization mechanisms on a shaft thereof such that the trocar provides a stable delivery mechanism to the injection device. The trocar is configured to receive an injection needle therein that is attached to the injection device, and the injection device can deliver fluid therethrough to a target tissue site.

### FIELD

The present disclosure relates generally to devices, methods, and systems for delivering therapeutic agents such as compounds, compositions, cells, or cellular products such as exosomes. In a non-limiting example, devices provided herein have metered infusion capabilities and are configured to deliver agents extravascularly to target locations within a patient while maintaining positional stability.

### BACKGROUND

Cell therapy is a therapy process in which cellular material is injected into a patient for a therapeutic effect to treat a variety of different diseases, especially to target select organs within a body of a patient. In some cell therapy, cellular material can be extracted from a patient, processed for therapeutic effect, and reinjected into the patient at a treatment or delivery site. In certain cases, for the delivery of cells to be successful the cells being injected typically must also be intact living cells. Delivery of cell therapies can be accomplished in several different ways, such as through an intravascular delivery or an extravascular delivery. Intravascular delivery involves a cell therapy that is infused through vascular access. Targeting certain organs for treatment through this process can be accomplished in a variety of ways. However, the efficiency can be low, and residence time of therapeutic cellular material provided to the organ(s) can be short due to flushing that a patient's body performs naturally.

### BRIEF SUMMARY

Provided herein are, inter alia, devices, methods, and systems for delivering therapeutic agents such as compounds, compositions, cells, and cellular products. In certain embodiments, devices and systems provided herein are configured for the administration of cell therapy. In certain embodiments, the devices and systems provided herein include metered infusion capabilities. In certain embodiments, the devices and systems provided herein are configured to deliver a therapeutic agent extravascularly to a target location within a patient, such as the interior of an organ, while maintaining positional stability. For example in one exemplary embodiment, a cell therapy delivery device is provided that includes a body having an actuator, a fluid reservoir with fluid therein, and a fluid delivery mechanism. In certain embodiments, a detachable injection needle extends distally from the body, and the fluid delivery mechanism is configured to deliver a continuous stream or boluses of the fluid through the injection needle. In certain embodiments, the organ is a kidney. In certain embodiments, the patient has cancer and the organ comprises a tumor.

The device can have numerous variations. In certain embodiments, the fluid delivery mechanism can include an electromechanical system having a central processing unit and a pump. In certain embodiments, the device can also include a valve that is configured to translate proximally and distally parallel to the injection needle during placement of the device and delivery of the fluid. In certain embodiments, the valve can be configured to translate about 2 cm distally and proximally. In certain embodiments, the device can include a fluid receiver configured to removably and replaceably receive the fluid reservoir therein. In certain embodiments, the fluid reservoir can include at least one cartridge that includes a known dosage of a fluid. In certain embodiments, the fluid receiver can be configured to receive one or more (e.g., 1, 2, 3, 4, or 5) cartridges aseptically. In certain embodiments, the fluid receiver can be configured to receive a multiple cartridges serially (e.g., the contents of one cartridge are used, the cartridge is removed and then one or more additional cartridges are inserted as needed to continue dosing). In certain embodiments, the fluid receiver can be configured to receive a plurality of cartridges concurrently. In certain embodiments, the fluid can include therapeutic cells or cellular products for the treatment of kidney disease. In certain embodiments, the fluid can included an anti-cancer agent for the treatment of cancer. In certain embodiments, the device can include a touch display that can be configured to control operation of the device. In certain embodiments, the display can be configured to set one or more parameters for delivery of the fluid, including at least one of pressure and volume. In certain embodiments, the display can be configured to provide real-time dispensing information of the fluid during delivery. In certain embodiments, the actuator can be one of a trigger, a plunger, a switch, or a button. In certain embodiments, the device can also include an engagement feature on a distal end of the body configured to detachably engage a trocar.

In an aspect, a trocar is provided that includes an elongate body with proximal and distal ends. In certain embodiments, the body has a head on the proximal end thereof, an elongate shaft extending distally from the head, and a lumen extending from the proximal end to the distal end therethrough. In certain embodiments, a stabilizing means is provided on a distal portion of the elongate shaft and is configured to stabilize the distal end of the elongate body relative to a tissue surface.

The trocar can have numerous variations. In certain embodiments, the stabilizing means can include one or more engagement components that are configured to deploy to releasably grasp the tissue surface upon actuation. In certain embodiments, the engagement components can include a plurality of feet. In certain embodiments, the feet can have micro-hooks thereon. In certain embodiments, the engagement components can include at least one of an adhesive component, a suction component, and a pincher component. In certain embodiments, the trocar can include a removable stylet configured to extend through the lumen of the elongate body. In certain embodiments, the stylet is configured to actuate the engagement components upon removal. In certain embodiments, at least part of the elongate shaft can be configured to translate distally and proximally parallel to a longitudinal axis of the elongate shaft. In certain embodiments, the at least part of the elongate shaft can be configured to translate about 2 cm distally and proximally.

In an aspect, a method of delivering a pharmaceutical fluid formulation to tissue is provided that includes attaching an injection device to a trocar. In certain embodiments, the trocar has a lumen therethrough and a stylet positioned therein. In certain embodiments, the method also includes connecting a fluid source to the injection device, and advancing the injection device and trocar through an outer tissue surface of a patient and penetrating an inner tissue target site. In certain embodiments, the method further includes removing the stylet from the trocar and disengaging the injection device and the trocar, and attaching an injection needle to the injection device. In certain embodiments, the method also includes inserting the injection needle through the trocar to the tissue target site, and actuating the injection device to deliver a continuous stream or boluses of a fluid from the fluid source through the injection needle and to the tissue target site. In certain embodiments, the pharmaceutical fluid formulation comprises, consists essentially of, or consists of a population of cells or a product thereof a fluid pharmaceutically acceptable carrier. In certain embodiments, the cell therapy comprises stem cells, progenitor cells, primary cells, or a cell line. In certain embodiments, the tissue target site is a kidney. In certain embodiments, the patient has a kidney disease. In certain embodiments, the kidney disease is chronic kidney disease. In certain embodiments, the cell therapy comprises bioactive renal cells. In certain embodiments, the cell therapy comprises selected renal cells. In certain embodiments, the cell therapy comprises a liquid formulation comprising cells and a temperature-sensitive biomaterial. In certain embodiments, the cell therapy is Neo-Kidney Augment (NKA). In certain embodiments, the cells are in the form of spheroids or cell clusters. In certain embodiments, the pharmaceutical fluid formulation comprises a cell product, such as a vesicle, e.g., a microvesicle or an exosome. In certain embodiments, the pharmaceutical fluid formulation comprises a compound. In certain embodiments, the pharmaceutical fluid formulation comprises an anti-cancer agent. In certain embodiments, the patient has cancer. In certain embodiments, the tissue target site is a tumor.

The method can vary in numerous ways. For example, the method can further include, prior to inserting the injection needle through the trocar, deploying a stabilizing means on a distal portion of the trocar to stabilize the distal end of the trocar relative to the tissue target site. In certain embodiments, deploying the stabilizing means can be actuated by removal of the stylet. In certain embodiments, the method can also include retracting the injection needle during delivery of the fluid (e.g., a continuous stream or boluses thereof). In certain embodiments, the method can further include, during actuating the injection device, stabilizing the injection device using a translating valve and stabilizing the trocar using a compressive spring section of the trocar.

### BRIEF DESCRIPTION OF THE DRAWINGS

The invention will be more fully understood from the following detailed description taken in conjunction with the accompanying drawings, in which:

FIG. 1 illustrates a view of a patient in a prone position:

FIG. 2 illustrates a view of a patient in a lateral position:

FIG. 3 illustrates a diagram detailing an exemplary process for using commercially available devices to treat a subject:

FIG. 4A illustrates an embodiment of the use of commercially available devices to deliver treatment to a patient in accordance with the process of FIG. 3:

FIGS. 4B and C illustrate embodiments of commercially available devices for delivering treatment to a patient in accordance with the process of FIG. 3:

FIG. 5 illustrates a side view of one embodiment of a trocar:

FIG. 6 illustrates a side view of one embodiment of a cannula:

FIG. 7 illustrates a side view of one embodiment of an injection device with a trocar attached thereto that has a stylet inserted therein:

FIG. 8 illustrates a simplified diagram of a bolus fluid delivery profile:

FIG. 9 illustrates a simplified diagram of a continuous fluid delivery profile:

FIG. 10 illustrates one embodiment of a cartridge:

FIG. 11 illustrates a side view of the trocar of FIG. 7:

FIG. 12 illustrates a side view of the trocar of FIG. 7 being deployed:

FIG. 13 illustrates a side view of an embodiment of a trocar being inserted:

FIG. 14 illustrates a side view of the trocar of FIG. 13 being deployed:

FIG. 15 illustrates a side view of the trocar of FIG. 13 being deployed:

FIG. 16A illustrates a side view of one embodiment of a drywall anchor being deployed:

FIG. 16B illustrates a side view of the drywall anchor of FIG. 16A being deployed:

FIG. 16C illustrates a side view of the drywall anchor of FIG. 16B being deployed:

FIG. 16D illustrates a side view of the drywall anchor of FIG. 16C being deployed:

FIG. 17 illustrates a distal view of one embodiment of a needle with surface features thereon:

FIG. 18A illustrates a distal tip view of one embodiment of a stylet:

FIG. 18B illustrates a distal tip view of another embodiment of a stylet:

FIG. 18C illustrates a distal tip view of another embodiment of a stylet:

FIG. 18D illustrates a distal tip view of another embodiment of a stylet:

FIG. 19 illustrates a simplified diagram of one embodiment of an injection needle:

FIG. 20 illustrates a cutaway side view of a renal capsule:

FIG. 21 illustrates a diagram addressing finding an optimum needle size:

FIG. 22 illustrates a distal end of an injection needle with a ruler;

FIG. 23 illustrates the distal end of the injection needle of FIG. 22 with a ruler highlighting placement of a hole in the distal end;

FIG. 24 illustrates an exemplary method of holding a small syringe for better control:

FIG. 25 illustrates a simplified cross-sectional view of placement of the trocar of FIG. 7 in a kidney:

FIG. 26 illustrates a simplified cross-sectional view of placement of the trocar of FIG. 7 in the kidney of FIG. 25;

FIGS. 27A-F illustrate an embodiment of delivering multiple boluses of a therapeutic agent into a kidney.

It should be understood that the above-referenced drawings are not necessarily to scale, presenting a somewhat simplified representation of various preferred features illustrative of the basic principles of the disclosure. The specific design features of the present disclosure, including, for example, specific dimensions, orientations, locations, and shapes, will be determined in part by the particular intended application and use environment.

### DETAILED DESCRIPTION

Hereinafter, embodiments of the present disclosure will be described in detail with reference to the accompanying drawings. As those skilled in the art would realize, the described embodiments may be modified in various different ways, all without departing from the spirit or scope of the present disclosure. Further, throughout the specification, like reference numerals refer to like elements.

The terminology used herein is for the purpose of describing particular embodiments only and is not intended to be limiting of the disclosure. As used herein, the singular forms “a,” “an,” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Linking terms such as “coupled,” “engaged,” etc. denote a physical relationship between two components whereby the components are either directly connected to one another or indirectly connected via one or more intermediary components.

It is understood that the term “patient” or other similar term as used herein is inclusive of any subject-human or animal-on which a therapy disclosed herein could be performed. The term “user” as used herein is inclusive of any entity capable of interacting with or controlling a device. The “user” may also be the “patient,” or the “user” and “patient” may be separate entities, as described herein. In certain embodiments, a subject is a living animal. In certain embodiments, a subject is a mammal such as a dog, cat, horse, rabbit, zoo animal, cow, pig, sheep, goat, camel, mouse, rat, or guinea pig. In certain embodiments, a subject is a primate such as a human, a chimpanzee, an orangutan, a monkey, or a baboon. In certain embodiments, a subject is a human. In certain embodiments, a subject is a patient, eligible for treatment, who is experiencing or has experienced one or more signs, symptoms, or other indicators of a kidney disease. Such subjects include without limitation subjects who are newly diagnosed or previously diagnosed and are now experiencing a recurrence or relapse, or are at risk for a kidney disease, no matter the cause. In certain embodiments, the subject may have been previously treated for a kidney disease, or not so treated. In certain embodiments, the subject has diabetes. In certain embodiments, the subject has Type I diabetes. In certain embodiments, the subject has Type II diabetes. In certain embodiments, the subject has chronic kidney disease. In certain embodiments, a subject has a congenital anomaly of a kidney and/or urinary tract. In certain embodiments, a subject is a human with congenital anomalies of the kidney and urinary tract. In certain embodiments, a subject is experiencing or has experienced one or more signs, symptoms, or other indicators of an organ-related disease, such as kidney disease, anemia, or erythropoietin (EPO) deficiency. In certain embodiments, the subject does not have diabetes. In certain embodiments, the subject does not have Type I diabetes. In certain embodiments, the subject does not have Type II diabetes. In certain embodiments, the subject does not have a kidney disease. In certain embodiments, the subject has cancer. In certain embodiments, the cancer comprises a solid tumor.

In an aspect, devices, systems, and methods provided herein are useful for administering an anti-cancer agent or chemotherapy to a patient. In certain embodiments, administering the anti-cancer agent comprises delivering the anti-cancer agent or chemotherapy into an internal tissue site or organ of the subject. In certain embodiments, a patient has a solid tumor. In certain embodiments, the solid tumor is within, on, invading, or part of an organ. In certain embodiments, the internal tissue site or organ is a kidney, lung, heart, spleen, stomach, pancreas, urinary bladder, brain, small intestine, colon, rectum, appendix, ovary, uterus, esophagus, liver, gallbladder, thyroid gland, parathyroid gland, adrenal gland, breast, lymph node, muscle, spinal cord, testicle, prostate, pharynx, larynx, bone, or trachea. In certain embodiments, the subject has cancer. In certain embodiments, the cancer is a melanoma (e.g., metastatic melanoma that has spread to an internal site such as an organ), a neuroendocrine tumor, a carcinoma, or a sarcoma. In certain embodiments, a patient has a sarcoma, bladder cancer, bone cancer, brain tumor, cervical cancer, colon cancer, esophageal cancer, gastric cancer, head and neck cancer, kidney cancer, myeloma, thyroid cancer, leukemia, prostate cancer, breast cancer (e.g. triple negative, estrogen receptor (ER) positive, ER negative, chemotherapy resistant, herceptin resistant, HER2 positive, doxorubicin resistant, tamoxifen resistant, ductal carcinoma, lobular carcinoma, primary, or metastatic breast cancer), ovarian cancer, pancreatic cancer, liver cancer (e.g. hepatocellular carcinoma), lung cancer (e.g. non-small cell lung carcinoma, squamous cell lung carcinoma, adenocarcinoma, large cell lung carcinoma, small cell lung carcinoma, carcinoid, or sarcoma), glioblastoma multiforme, glioma, melanoma, prostate cancer, castration-resistant prostate cancer, glioblastoma, ovarian cancer, lung cancer, squamous cell carcinoma (e.g., head, neck, or esophagus), or colorectal cancer. In certain embodiments, a subject has cancer of the thyroid, endocrine system, brain, breast, cervix, colon, head & neck, esophagus, liver, kidney, lung, non-small cell lung, melanoma, sarcoma, stomach, uterus, medulloblastoma, neuroblastoma, glioma, glioblastoma multiforme, ovarian cancer, rhabdomyosarcoma, primary thrombocytosis, primary macroglobulinemia, a primary brain tumor, malignant pancreatic insulanoma, malignant carcinoid, urinary bladder cancer, testicular cancer, thyroid cancer, esophageal cancer, genitourinary tract cancer, malignant hypercalcemia, endometrial cancer, adrenal cortical cancer, medullary thyroid cancer, medullary thyroid carcinoma, metastatic melanoma (e.g., melanoma that has spread to an internal site such as an organ), colorectal cancer, papillary thyroid cancer, hepatocellular carcinoma, Paget's disease of the nipple, a phyllodes tumor, lobular carcinoma, ductal carcinoma, cancer of pancreatic stellate cells, cancer of the hepatic stellate cells, or prostate cancer. The term “sarcoma” generally refers to a tumor which is made up of a substance like the embryonic connective tissue and is generally composed of closely packed cells embedded in a fibrillar or homogeneous substance. Sarcomas that may be treated using a device, system, or method provided herein include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma. The term “melanoma” is taken to mean a tumor arising from the melanocytic system of the skin and other organs. Melanomas that may be treated using a devise, system, or method provided herein include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, or superficial spreading melanoma. In certain embodiments, the melanoma is a metastatic melanoma that has spread to an internal site (such as an organ or lymph node) of a patient. The term “carcinoma” refers to a malignant new growth made up of epithelial cells tending to infiltrate the surrounding tissues and give rise to metastases. Exemplary carcinomas that may be treated using a device, system, or method provided herein include, for example, medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, ductal carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lobular carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tubular carcinoma, tuberous carcinoma, verrucous carcinoma, or carcinoma villosum.

An “anti-cancer agent” is a therapeutic used in the treatment or prevention of cancer. In certain embodiments, an anti-cancer agent can be a large molecule (e.g., a protein or other organic compound having a molecular weight of at least 2000 daltons) or small molecule (e.g., an organic compound having a molecular weight less than 2000 daltons). Example anti-cancer agents include antibodies, small molecules, and large molecules or combinations thereof. In certain embodiments, an anti-cancer agent comprises a cell, such as an immune cell. In certain embodiments, the immune cell has been modified (e.g., genetically and/or via exposure to a tumor antigen) to attack or promote an immune response to tumor cells. In certain embodiments, the immune cell is a T cell (such as a CD4 T cell, a CD8 T cell or a combination thereof) or a dendritic cell (such as a plasmacytoid dendritic cell). In certain embodiments, the immune cell has been genetically modified, such as a chimeric antigen receptor (CAR) T cell. In certain embodiments, the anti-cancer agent inhibits the growth or proliferation of cells. In certain embodiments, an anti-cancer agent is a chemotherapeutic. In certain embodiments, an anti-cancer agent is an agent identified herein having utility in methods of treating cancer. In certain embodiments, an anticancer agent is an agent approved by the United States Food and Drug Administration (FDA) or similar regulatory agency of a country other than the United States, for treating cancer. Examples of anti-cancer agents include, but are not limited to, MEK (e.g. MEK1, MEK2, or MEK1 and MEK2) inhibitors (e.g. XL518, CI-1040, PD035901, selumetinib/AZD6244, GSK1120212/trametinib, GDC-0973, ARRY-162, ARRY-300, AZD8330, PD0325901, U0126, PD98059, TAK-733, PD318088, AS703026, BAY 869766), alkylating agents (e.g., cyclophosphamide, ifosfamide, chlorambucil, busulfan, melphalan, mechlorethamine, uramustine, thiotepa, nitrosoureas, nitrogen mustards (e.g., mechloroethamine, cyclophosphamide, chlorambucil, meiphalan), ethylenimine and methylmelamines (e.g., hexamethlymelamine, thiotepa), alkyl sulfonates (e.g., busulfan), nitrosoureas (e.g., carmustine, lomusitne, semustine, streptozocin), triazenes (decarbazine)), anti-metabolites (e.g., 5-azathioprine, leucovorin, capecitabine, fludarabine, gemcitabine, pemetrexed, raltitrexed, folic acid analog (e.g., methotrexate), or pyrimidine analogs (e.g., fluorouracil, floxouridine, cytarabine), purine analogs (e.g., mercaptopurine, thioguanine, pentostatin), etc.), plant alkaloids (e.g., vincristine, vinblastine, vinorelbine, vindesine, podophyllotoxin, paclitaxel, docetaxel, etc.), topoisomerase inhibitors (e.g., irinotecan, topotecan, amsacrine, etoposide (VP16), etoposide phosphate, teniposide, etc.), antitumor antibiotics (e.g., doxorubicin, adriamycin, daunorubicin, epirubicin, actinomycin, bleomycin, mitomycin, mitoxantrone, plicamycin, etc.), platinum-based compounds or platinum containing agents (e.g. cisplatin, oxaloplatin, carboplatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide), epipodophyllotoxins (e.g., etoposide), antibiotics (e.g., daunorubicin, doxorubicin, bleomycin), enzymes (e.g., L-asparaginase), inhibitors of mitogen-activated protein kinase signaling (e.g. U0126, PD98059, PD184352, PD0325901, ARRY-142886, SB239063, SP600125, BAY 43-9006, wortmannin, or LY294002, Syk inhibitors, mTOR inhibitors, antibodies (e.g., rituxan), gossyphol, genasense, polyphenol E, Chlorofusin, all trans-retinoic acid (ATRA), bryostatin, tumor necrosis factor-related apoptosis-inducing ligand (TRAIL), 5-aza-2′-deoxycytidine, all trans retinoic acid, doxorubicin, vincristine, etoposide, gemcitabine, imatinib (Gleevec R), geldanamycin, 17-N-Allylamino-17-Demethoxygeldanamycin (17-AAG), flavopiridol, LY294002, bortezomib, trastuzumab, BAY 11-7082, PKC412, PD184352, 20-epi-1, 25 dihydroxyvitamin D3: 5-ethynyluracil: abiraterone: aclarubicin: acylfulvene; adecypenol: adozelesin: aldesleukin: ALL-TK antagonists: altretamine; ambamustine; amidox: amifostine: aminolevulinic acid; amrubicin: amsacrine: anagrelide; anastrozole: andrographolide: angiogenesis inhibitors: antagonist D: antagonist G; antarelix: anti-dorsalizing morphogenetic protein-1: antiandrogen, prostatic carcinoma: antiestrogen: antineoplaston: antisense oligonucleotides: aphidicolin glycinate: apoptosis gene modulators: apoptosis regulators: apurinic acid: ara-CDP-DL-PTBA; arginine deaminase: asulacrine: atamestane; atrimustine; axinastatin 1; axinastatin 2; axinastatin 3; azasetron: azatoxin; azatyrosine: baccatin III derivatives; balanol: batimastat: BCR/ABL antagonists: benzochlorins: benzoylstaurosporine: beta lactam derivatives: beta-alethine: betaclamycin B: betulinic acid: bFGF inhibitor; bicalutamide; bisantrene: bisaziridinylspermine; bisnafide: bistratene A: bizelesin; breflate: bropirimine: budotitane; buthionine sulfoximine: calcipotriol: calphostin C; camptothecin derivatives: canarypox IL-2: capecitabine: carboxamide-amino-triazole; carboxyamidotriazole: CaRest M3: CARN 700; cartilage derived inhibitor; carzelesin; casein kinase inhibitors (ICOS): castanospermine: cecropin B: cetrorelix: chlorins; chloroquinoxaline sulfonamide: cicaprost: cis-porphyrin: cladribine: clomifene analogues: clotrimazole: collismycin A: collismycin B: combretastatin A4; combretastatin analogue: conagenin: crambescidin 816: crisnatol: cryptophycin 8; cryptophycin A derivatives: curacin A: cyclopentanthraquinones: cycloplatam; cypemycin: cytarabine ocfosfate: cytolytic factor: cytostatin: dacliximab; decitabine; dehydrodidemnin B: deslorelin: dexamethasone; dexifosfamide: dexrazoxane; dexverapamil: diaziquone: didemnin B: didox: diethylnorspermine: dihydro-5-azacytidine: 9-dioxamycin: diphenyl spiromustine: docosanol; dolasetron; doxifluridine: droloxifene: dronabinol: duocarmycin SA: ebselen: ecomustine; edelfosine: edrecolomab; eflornithine: elemene; emitefur: epirubicin: epristeride; estramustine analogue: estrogen agonists: estrogen antagonists: etanidazole: etoposide phosphate: exemestane: fadrozole; fazarabine: fenretinide: filgrastim; finasteride; flavopiridol: flezelastine: fluasterone: fiudarabine: fluorodaunorunicin hydrochloride; forfenimex: formestane; fostriecin: fotemustine: gadolinium texaphyrin: gallium nitrate: galocitabine: ganirelix: gelatinase inhibitors: gemcitabine: glutathione inhibitors: hepsulfam; heregulin: hexamethylene bisacetamide; hypericin: ibandronic acid: idarubicin; idoxifene; idramantone; ilmofosine; ilomastat; imidazoacridones; imiquimod: immunostimulant peptides: insulin-like growth factor-1 receptor inhibitor: interferon agonists: interferons: interleukins: iobenguane; iododoxorubicin; ipomeanol, 4-: iroplact: irsogladine: isobengazole: isohomohalicondrin B: itasetron; jasplakinolide: kahalalide F: lamellarin-N triacetate: lanreotide: leinamycin; lenograstim: lentinan sulfate: leptolstatin: letrozole: leukemia inhibiting factor; leukocyte alpha interferon: leuprolide+estrogen+progesterone: leuprorelin; levamisole: liarozole: linear polyamine analogue: lipophilic disaccharide peptide: lipophilic platinum compounds: lissoclinamide 7: lobaplatin: lombricine; lometrexol; lonidamine: losoxantrone: lovastatin; loxoribine; lurtotecan: lutetium texaphyrin; lysof lline: lytic peptides: maitansine: mannostatin A: marimastat: masoprocol; maspin: matrilysin inhibitors: matrix metalloproteinase inhibitors: menogaril; merbarone: meterelin: methioninase: metoclopramide: MIF inhibitor; mifepristone; miltefosine: mirimostim: mismatched double stranded RNA: mitoguazone: mitolactol; mitomycin analogues: mitonafide: mitotoxin fibroblast growth factor-saporin; mitoxantrone: mofarotene: molgramostim: monoclonal antibody, human chorionic gonadotrophin: monophosphoryl lipid A+myobacterium cell wall sk: mopidamol; multiple drug resistance gene inhibitor: multiple tumor suppressor 1-based therapy; mustard anticancer agent: mycaperoxide B: mycobacterial cell wall extract; myriaporone: N-acetyldinaline: N-substituted benzamides: nafarelin: nagrestip; naloxone+pentazocine: napavin: naphterpin: nartograstim: nedaplatin: nemorubicin; neridronic acid: neutral endopeptidase: nilutamide: nisamycin: nitric oxide modulators; nitroxide antioxidant: nitrullyn: 06-benzyl guanine: octreotide; okicenone: oligonucleotides: onapristone: ondansetron: ondansetron: oracin; oral cytokine inducer: ormaplatin: osaterone: oxaliplatin: oxaunomycin: palauamine; palmitoylrhizoxin: pamidronic acid: panaxytriol: panomifene: parabactin; pazelliptine: pegaspargase: peldesine: pentosane polysulfate sodium: pentostatin; pentrozole: perflubron: perfosfamide: perillyl alcohol: phenazinomycin: phenyl acetate: phosphatase inhibitors: picibanil: pilocarpine hydrochloride: pirarubicin; piritrexim: placetin A: placetin B: plasminogen activator inhibitor: platinum complex; platinum compounds: platinum-triamine complex: porfimer sodium: porfiromycin; prednisone: propyl bis-acridone: prostaglandin J2: proteasome inhibitors: protein A-based immune modulator: protein kinase C inhibitor: protein kinase C inhibitors, microalgal: protein tyrosine phosphatase inhibitors: purine nucleoside phosphorylase inhibitors: purpurins: pyrazoloacridine: pyridoxylated hemoglobin polyoxyethylerie conjugate: raf antagonists: raltitrexed: ramosetron: ras farnesyl protein transferase inhibitors: ras inhibitors: ras-GAP inhibitor: retelliptine demethylated: rhenium Re 186 etidronate: rhizoxin: ribozymes: RII retinamide: rogletimide: rohitukine; romurtide: roquinimex: rubiginone B 1: ruboxyl: safingol: saintopin: SarCNU; sarcophytol A: sargramostim: Sdi 1 mimetics: semustine; senescence derived inhibitor 1: sense oligonucleotides: signal transduction inhibitors: signal transduction modulators: single chain antigen-binding protein: sizofuran: sobuzoxane: sodium borocaptate: sodium phenylacetate: solverol: somatomedin binding protein: sonermin; sparfosic acid: spicamycin D: spiromustine: splenopentin: spongistatin 1: squalamine; stem cell inhibitor: stem-cell division inhibitors: stipiamide: stromelysin inhibitors; sulfinosine: superactive vasoactive intestinal peptide antagonist: suradista: suramin; swainsonine: synthetic glycosaminoglycans: tallimustine: tamoxifen methiodide; tauromustine: tazarotene: tecogalan sodium: tegafur: tellurapyrylium; telomerase inhibitors: temoporfin: temozolomide: teniposide: tetrachlorodecaoxide: tetrazomine; thaliblastine: thiocoraline: thrombopoietin: thrombopoietin mimetic: thymalfasin; thymopoietin receptor agonist: thymotrinan: thyroid stimulating hormone: tin ethyl etiopurpurin: tirapazamine: titanocene bichloride: topsentin: toremifene: totipotent stem cell factor: translation inhibitors: tretinoin: triacetyluridine; triciribine; trimetrexate: triptorelin: tropisetron: turosteride: tyrosine kinase inhibitors; tyrphostins: UBC inhibitors: ubenimex: urogenital sinus-derived growth inhibitory factor: urokinase receptor antagonists: vapreotide: variolin B: vector system, erythrocyte gene therapy: velaresol: veramine: verdins: verteporfin: vinorelbine; vinxaltine: vitaxin: vorozole; zanoterone: zeniplatin; zilascorb: zinostatin stimalamer; Adriamycin: Dactinomycin: Bleomycin: Vinblastine: Cisplatin: acivicin; aclarubicin; acodazole hydrochloride: acronine: adozelesin: aldesleukin: altretamine: ambomycin; ametantrone acetate: aminoglutethimide: amsacrine: anastrozole: anthramycin; asparaginase: asperlin: azacitidine: azetepa: azotomycin: batimastat: benzodepa; bicalutamide: bisantrene hydrochloride: bisnafide dimesylate: bizelesin: bleomycin sulfate: brequinar sodium: bropirimine: busulfan: cactinomycin: calusterone; caracemide: carbetimer: carboplatin: carmustine; carubicin hydrochloride: carzelesin; cedefingol: chlorambucil: cirolemycin; cladribine: crisnatol mesylate; cyclophosphamide: cytarabine: dacarbazine: daunorubicin hydrochloride; decitabine; dexormaplatin: dezaguanine: dezaguanine mesylate: diaziquone: doxorubicin; doxorubicin hydrochloride: droloxifene; droloxifene citrate: dromostanolone propionate: duazomycin: edatrexate: eflornithine hydrochloride: elsamitrucin; enloplatin: enpromate: epipropidine: epirubicin hydrochloride: erbulozole: esorubicin hydrochloride: estramustine: estramustine phosphate sodium: etanidazole: etoposide; etoposide phosphate: etoprine: fadrozole hydrochloride; fazarabine: fenretinide; floxuridine: fludarabine phosphate: fluorouracil: fluorocitabine: fosquidone; fostriecin sodium: gemcitabine: gemcitabine hydrochloride: hydroxyurea: idarubicin hydrochloride: ifosfamide: iimofosine: interleukin II (including recombinant interleukin II, or rIL2), interferon alfa-2a: interferon alfa-2b: interferon alfa-nl; interferon alfa-n3: interferon beta-1a: interferon gamma-1b: iproplatin: irinotecan hydrochloride: lanreotide acetate: letrozole: leuprolide acetate: liarozole hydrochloride: lometrexol sodium: lomustine: losoxantrone hydrochloride; masoprocol: maytansine: mechlorethamine hydrochloride: megestrol acetate; melengestrol acetate: melphalan: menogaril: mercaptopurine: methotrexate; methotrexate sodium: metoprine: meturedepa: mitindomide: mitocarcin: mitocromin; mitogillin: mitomalcin: mitomycin: mitosper; mitotane: mitoxantrone hydrochloride; mycophenolic acid: nocodazoie: nogalamycin: ormaplatin: oxisuran; pegaspargase; peliomycin: pentamustine: peplomycin sulfate: perfosfamide: pipobroman; piposulfan: piroxantrone hydrochloride: plicamycin: plomestane: porfimer sodium; porfiromycin: prednimustine: procarbazine hydrochloride: puromycin: puromycin hydrochloride: pyrazofurin: riboprine: rogletimide: safingol: safingol hydrochloride; semustine: simtrazene: sparfosate sodium: sparsomycin: spirogermanium hydrochloride: spiromustine: spiroplatin: streptonigrin: streptozocin: sulofenur; talisomycin: tecogalan sodium: tegafur: teloxantrone hydrochloride: temoporfin; teniposide: teroxirone: testolactone: thiamiprine: thioguanine: thiotepa: tiazofurin; tirapazamine: toremifene citrate: trestolone acetate: triciribine phosphate; trimetrexate: trimetrexate glucuronate: triptorelin: tubulozole hydrochloride; uracil mustard: uredepa: vapreotide: verteporfin: vinblastine sulfate; vincristine sulfate; vindesine: vindesine sulfate: vinepidine sulfate: vinglycinate sulfate: vinleurosine sulfate: vinorelbine tartrate: vinrosidine sulfate: vinzolidine sulfate: vorozole; zeniplatin: zinostatin: zorubicin hydrochloride, agents that arrest cells in the G2-M phases and/or modulate the formation or stability of microtubules, (e.g. Taxol™ (i.e. paclitaxel), Taxotere™, compounds comprising the taxane skeleton, Erbulozole (i.e. R-55104), Dolastatin 10 (i.e. DLS-10 and NSC-376128), Mivobulin isethionate (i.e. as CI-980), Vincristine, NSC-639829, Discodermolide (i.e. as NVP-XX-A-296), ABT-751 (Abbott, i.e. E-7010), Altorhyrtins (e.g. Altorhyrtin A and Altorhyrtin C), Spongistatins (e.g. Spongistatin 1, Spongistatin 2, Spongistatin 3, Spongistatin 4, Spongistatin 5, Spongistatin 6, Spongistatin 7, Spongistatin 8, and Spongistatin 9), Cemadotin hydrochloride (i.e. LU-103793 and NSC-D-669356), Epothilones (e.g. Epothilone A, Epothilone B, Epothilone C (i.e. desoxyepothilone A or dEpoA), Epothilone D (i.e. KOS-862, dEpoB, and desoxyepothilone B), Epothilone E, Epothilone F, Epothilone B N-oxide, Epothilone A N-oxide, 16-aza-epothilone B, 21-aminoepothilone B (i.e. BMS-310705), 21-hydroxyepothilone D (i.e. Desoxyepothilone F and dEpoF), 26-fluoroepothilone, Auristatin PE (i.e. NSC-654663), Soblidotin (i.e. TZT-1027), Vincristine sulfate, Cryptophycin 52 (i.e. LY-355703), Vitilevuamide, Tubulysin A, Canadensol, Centaureidin (i.e. NSC-106969), Oncocidin Al (i.e. BTO-956 and DF E), Fijianolide B, Laulimalide, Narcosine (also known as NSC-5366), Nascapine, Hemiasterlin, Vanadocene acetyl acetonate, Monsatrol, Inanocine (i.e. NSC-698666), Eleutherobins (such as Desmethyleleutherobin, Desaetyleleutherobin, Isoeleutherobin A, and Z-Eleutherobin), Caribaeoside, Caribaeolin, Halichondrin B, Diazonamide A, Taccalonolide A, Diozostatin, (-)-Phenylahistin (i.e. NSCL-96F037), Myoseverin B, Resverastatin phosphate sodium, steroids (e.g., dexamethasone), finasteride, aromatase inhibitors, gonadotropin-releasing hormone agonists (GnRH) such as goserelin or leuprolide, adrenocorticosteroids (e.g., prednisone), progestins (e.g., hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), immunostimulants (e.g., Bacillus Calmette-Guerin (BCG), levamisole, interleukin-2, alpha-interferon, etc.), monoclonal antibodies (e.g., anti-CD20, anti-F¦R2, anti-CD52, anti-ULA-DR, and anti-VEGF monoclonal antibodies), immunotoxins (e.g., anti-CD33 monoclonal antibody-calicheamicin conjugate, anti-CD22 monoclonal antibody-pseudomonas exotoxin conjugate, etc.), radioimmunotherapy (e.g., anti-CD20 monoclonal antibody conjugated to Ulln, 90 Y, or ml, etc.), triptolide, homoharringtonine, dactinomycin, doxorubicin, epirubicin, topotecan, itraconazole, vindesine, cerivastatin, vincristine, deoxyadenosine, sertraline, pitavastatin, irinotecan, clofazimine, 5-nonyloxytryptamine, vemurafenib, dabrafenib, erlotinib, gefitinib, EGFR inhibitors, epidermal growth factor receptor (EGFR)-targeted therapy or therapeutic (e.g. gefitinib (Iressa™), erlotinib (Tarceva™), cetuximab (Erbitux™), lapatinib (Tykerb™), panitumumab (Vectibix™), vandetanib (Caprelsa™), afatinib/BIBW2992, CI-1033/canertinib, neratinib/HKI-272, CP-724714, TAK-285, AST-1306, ARRY334543, ARRY-380, AG-1478, dacomitinib/PF299804, OSI-420/desmethyl erlotinib, AZD8931, AEE788, pelitinib/EKB-569, CUDC-101, WZ8040, WZ4002, WZ3146, AG-490, XL647, PD153035, BMS-599626), sorafenib, imatinib, sunitinib, dasatinib, hormonal therapies, and the like.

The transitional term “comprising,” which is synonymous with “including,” “containing,” or “characterized by,” is inclusive or open-ended and does not exclude additional, unrecited features, integers, steps, operations, elements, and/or components. By contrast, the transitional phrase “consisting of” excludes any features, integers, steps, operations, elements, and/or components not specified. The transitional phrase “consisting essentially of′ limits the scope of a claim to the specified features, integers, steps, operations, elements, and/or components “and those that do not materially affect the basic and novel characteristic(s)” of the claimed invention.

As used herein, the term “about” in the context of a numerical value or range means±10% of the numerical value or range recited or claimed, unless the context requires a more limited range.

The term “ambient temperature” refers to the temperature at which the formulations of the present disclosure will be administered to a subject. Generally, the ambient temperature is the temperature of a temperature-controlled environment. Ambient temperature ranges from about 18° C. to about 30° C. In certain embodiments, ambient temperature is about 18° C., about 19° C., about 20° ° C., about 21° C., about 22° C., about 23° C., about 24° C., about 25° C., about 26° C., about 27° C., about 28° C., about 29° C., or about 30° C.

A “pharmaceutical fluid formulation” is a pharmaceutical composition that is a liquid at the time it is delivered (i.e., administered) to a patient. In certain embodiments, a pharmaceutical fluid formulation comprises an active agent such as a compound, cell, or cell product and a pharmaceutically acceptable carrier. In certain embodiments, the pharmaceutically acceptable carrier is a temperature-sensitive biomaterial.

Devices, systems, and methods provided herein are generally useful for the delivery of pharmaceutical fluid formulations to tissue sites such as organs (e.g., solid organs). In certain embodiments, the tissue site is a tumor (e.g., a solid or hard tumor). In certain embodiments, the tissue site is an organ that comprises cancer cells or a tumor. In certain embodiments, the tissue site comprises tumor cells. In certain embodiments, the tissue site is a lymph node that comprises tumor cells. However, in certain embodiments, the present subject matter is particularly useful for delivering bioactive renal cells (such as bioactive renal cells, e.g., selected renal cells) to kidneys of patients with a kidney disease.

The term “bioactive renal cells” or “BRCs” as used herein refers to renal cells having one or more of the following properties when administered into the kidney of a subject: capability to reduce (e.g., slow or halt) the worsening or progression of chronic kidney disease or a symptom thereof, capability to enhance renal function, capability to affect (improve) renal homeostasis, and capability to promote healing, repair and/or regeneration of renal tissue or kidney. In certain embodiments, these cells may include functional tubular cells (e.g., based on improvements in creatinine excretion and protein retention), glomerular cells (e.g., based on improvement in protein retention), vascular cells and other cells of the corticomedullary junction. In certain embodiments, BRCs are obtained from isolation and expansion of renal cells from kidney tissue. In certain embodiments, BRCs are obtained from isolation and expansion of renal cells from kidney tissue using methods that select for bioactive cells. In certain embodiments, the BRCs have a regenerative effect on the kidney. In certain embodiments, BRCs comprise, consist essentially of, or consist of selected renal cells (SRCs). In certain embodiments, BRCs are SRCs.

In certain embodiments, SRCs are cells obtained from isolation and expansion of renal cells from a suitable renal tissue source, wherein the SRCs contain a greater percentage of one or more cell types and lacks or has a lower percentage of one or more other cell types, as compared to a starting kidney cell population. In certain embodiments, the SRCs contain an increased proportion of BRCs compared to a starting kidney cell population. In certain embodiments, an SRC population is an isolated population of kidney cells enriched for specific bioactive components and/or cell types and/or depleted of specific inactive and/or undesired components or cell types for use in the treatment of kidney disease, i.e., providing stabilization and/or improvement and/or regeneration of kidney function. SRCs provide superior therapeutic and regenerative outcomes as compared with the starting population. In certain embodiments, SRCs are obtained from the patient's renal cortical tissue via a kidney biopsy. In certain embodiments, SRCs are selected (e.g., by fluorescence-activated cell sorting or “FACS”) based on their expression of one or more markers.

In certain embodiments, SRCs are depleted (e.g., by fluorescence-activated cell sorting or “FACS”) of one or more cell types based on the expression of one or more markers on the cell types. In certain embodiments, SRCs are selected from a population of bioactive renal cells. In certain embodiments, SRCs are selected by density gradient separation of expanded renal cells. In certain embodiments, SRCs are selected by separation of expanded renal cells by centrifugation across a density boundary, barrier, or interface, or single step discontinuous step gradient separation. In certain embodiments, SRCs are selected by continuous or discontinuous density gradient separation of expanded renal cells that have been cultured under hypoxic conditions. In certain embodiments, SRCs are selected by density gradient separation of expanded renal cells that have been cultured under hypoxic conditions for at least about 8, 12, 16, 20, or 24 hours. In certain embodiments, SRCs are selected by separation by centrifugation across a density boundary, barrier, or interface of expanded renal cells that have been cultured under hypoxic conditions. In certain embodiments, SRCs are selected by separation of expanded renal cells that have been cultured under hypoxic conditions for at least about 8, 12, 16, 20, or 24 hours by centrifugation across a density boundary, barrier, or interface (e.g., single-step discontinuous density gradient separation). In certain embodiments, SRCs are composed primarily of renal tubular cells. In certain embodiments, other parenchymal (e.g., vascular) and stromal (e.g., collecting duct) cells may be present in SRCs. In certain embodiments, less than about 10%, 9%, 8%, 7%, 6%, 5%, 4%, 3%, 2%, or 1% of the cells in a population of SRCs are vascular cells. In certain embodiments, less than about 10%, 9%, 8%, 7%, 6%, 5%, 4%, 3%, 2%, or 1% of the cells in a population of SRCs are collecting duct cells. In certain embodiments, less than about 10%, 9%, 8%, 7%, 6%, 5%, 4%, 3%, 2%, or 1% of the cells in a population of SRCs are vascular or collecting duct cells.

The term “Neo-Kidney Augment (NKA)” refers to a bioactive cell formulation which is an injectable product composed of autologous, selected renal cells (SRC) formulated in a biomaterial comprised of a gelatin-based hydrogel.

The term “kidney disease” as used herein refers to disorders associated with any stage or degree of acute or chronic renal failure that results in a loss of the kidney's ability to perform the function of blood filtration and elimination of excess fluid, electrolytes, and wastes from the blood. Kidney disease may also include endocrine dysfunctions such as anemia (erythropoietin-deficiency), and mineral imbalance (Vitamin D deficiency). Kidney disease may originate in the kidney or may be secondary to a variety of conditions, including (but not limited to) heart failure, hypertension, diabetes, autoimmune disease, or liver disease. Kidney disease may be a condition of chronic renal failure that develops after an acute injury to the kidney. For example, injury to the kidney by ischemia and/or exposure to toxicants may cause acute renal failure: incomplete recovery after acute kidney injury may lead to the development of chronic renal failure.

The term “spheroid” refers to an aggregate or assembly of cells cultured to allow 3-dimensional growth as opposed to growth as a monolayer. It is noted that the term “spheroid” does not imply that the aggregate is a geometric sphere. In certain embodiments, the aggregate may be highly organized with a well-defined morphology or the aggregate may be an unorganized mass. In certain embodiments, a spheroid may include a single cell type or more than one cell type. In certain embodiments, the cells may be primary isolates, or a permanent cell line, or a combination of the two. In certain embodiments, the spheroids (e.g., cellular aggregates or organoids) are formed in a spinner flask. In certain embodiments, the spheroids (e.g., cellular aggregates or organoids) are formed in a 3-dimensional matrix.

With respect to kidney disease and depending on context, “treatment” refers to both therapeutic treatment and prophylactic or preventative measures for kidney disease, tubular transport deficiency, or glomerular filtration deficiency wherein the object is to reverse, prevent or slow down (lessen) the targeted disorder or symptom. Those in need of treatment include those already having a kidney disease, tubular transport deficiency, or glomerular filtration deficiency as well as those prone to having a kidney disease, tubular transport deficiency, or glomerular filtration deficiency or those in whom the kidney disease, tubular transport deficiency, or glomerular filtration deficiency is to be prevented. In certain embodiments, treatment includes the stabilization and/or improvement of kidney function. With respect to cancer, treatment can include, e.g., reduced tumor volume, a reduced rate of tumor growth, an increased immune response to a tumor antigen, reduced cancer cell growth, reduced cancer cell proliferation, or reduced cancer cell survival (e.g., increased death such as apoptosis or necrosis of tumor cells).

### Administration of Therapeutic Agents

Extravascular delivery can involve direct injection of a pharmaceutical fluid formulation (e.g., a pharmaceutical fluid formulation comprising therapeutic cells) into an organ, such as into a stroma of the organ, via one or more devices, such as syringes, catheters, trocars, or the like. “Extravascular injection” means delivery by injection outside blood vessels. In extravascular delivery, residence time of therapeutic cells can be higher. For example, the clearing or flushing process can rely on clearing processes associated with local trauma and removal of edema at the delivery site. Delivery efficiency can also be high. However, successfully delivering therapeutic cells or products thereof can be difficult. For example, extravasation of the therapeutic cellular material through an entry hole of the delivery device can be an issue, caused by a variety of different problems. Extravasation can be considered to be the leakage (especially the unintended leakage) of a fluid out of a target injection site. For instance, natural motion of a delivery target due to a patient's movement (and thus movement of the target site), such as by respiration, can cause an unsteady delivery target. Inaccurate delivery of the therapeutic cellular material can also be caused by trauma to the treatment site due to impact and cutting by delivery instruments caused by movement during administration. Additionally, delivery can be difficult due to how infusion of therapeutic cells or products thereof occurs, such as by a continuous dosage stream to the target site.

Because of these and other reasons, improved devices, methods, and systems for delivering pharmaceutical fluid formulations (such as cell therapy) are needed. Provided herein are, inter alia, improved devices, methods, and systems for delivering therapeutic compositions such as compositions comprising compounds, cells, or cellular products.

In an aspect, included herein are devices, methods, and systems for delivering cell therapy, such as with metered infusion capabilities delivered extravascularly to target locations within a patient while maintaining positional stability. Using cell therapy has been a very popular and successful approach to treating a variety of different diseases. For example, extravascular delivery of cell therapy has been successful in providing high residence time of therapeutic cellular material at a treatment site and increased delivery efficiency. However, extravascular delivery has presented problems, such as extravasation of the therapeutic cellular material caused by factors like movement of a patient (and thus movement of the target site), trauma to the treatment site due to impact and cutting by delivery instruments caused by movement of the patient, and infusion of therapeutic cellular materials through a continuous dosage stream to the treatment site. Thus devices, methods, and systems are provided that are configured to provide stability to delivery of therapeutic agent (such as cellular material, e.g., cells or a product thereof such as exosomes), for example by providing a physically stable delivery process, and non-continuous dosage streams of therapeutic cellular material.

In an aspect, included herein are devices, methods, and systems for delivering a pharmaceutical fluid formulation (e.g., a cell therapy), such as with metered infusion capabilities delivered extravascularly to target locations within a patient while maintaining positional stability. Devices, systems, and methods provided herein may be used to treat a variety of different diseases, including but not limited to kidney diseases and cancer.

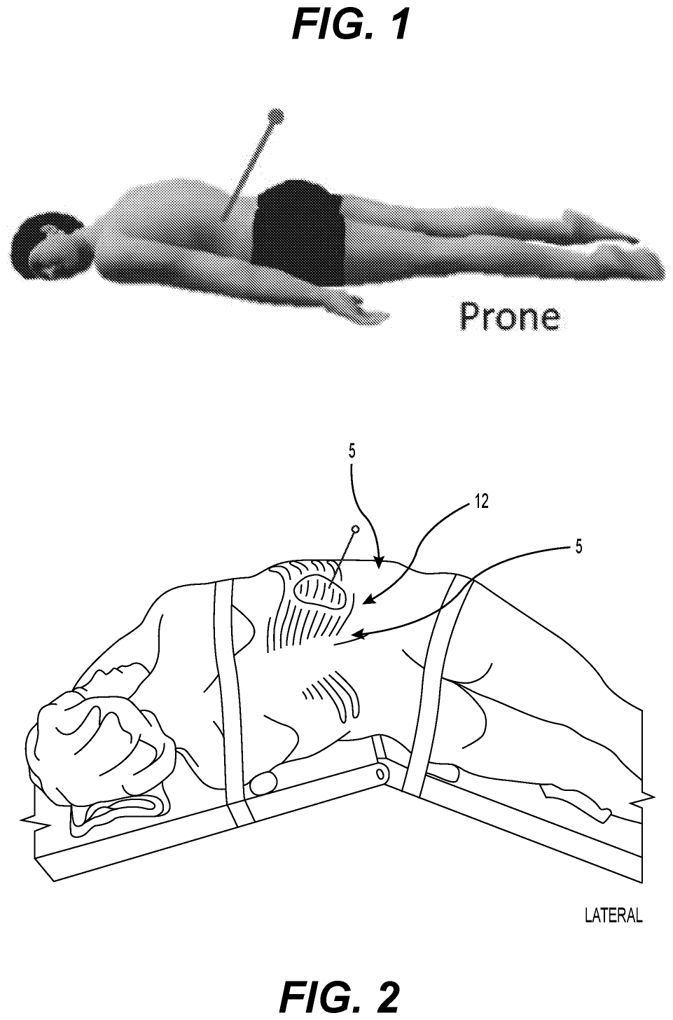

In certain embodiments, an injection device provided herein is configured to deliver a continuous stream of fluid to a target site. In certain embodiments, an injection device provided herein is configured to deliver a non-continuous stream or bolus of fluid to a target site. An exemplary device provided herein can be configured to deliver non-continuous streams or boluses of fluid, such as a pharmaceutical fluid formulation comprising cells or cell products, to a target site. A bolus can be considered to be a single physical portion of a pharmaceutical composition (such as a pharmaceutical fluid formulation). In certain embodiments, a bolus is a portion of a pharmaceutical composition (such as a pharmaceutical fluid formulation) that is delivered as a single event. In certain embodiments, a bolus is a portion of a pharmaceutical composition (such as a pharmaceutical fluid formulation) that is delivered as part of multiple portions (e.g., 2, 3, 4, 5, 6, 7, 8, 9, 10 or more portions) that are delivered one after the other, e.g., during administration of the pharmaceutical composition. In certain embodiments, a single bolus is delivered. In certain embodiments, no pharmaceutical composition is delivered between multiple discrete boluses. In certain embodiments, there is some continuous flow of pharmaceutical composition. In certain embodiments, delivery is continuous, but the amount of pharmaceutical composition is pulsed (i.e., flow does not stop, but increases and decreases over time). In certain embodiments, a device provided herein is configured for one-handed operation. In certain embodiments, the injection device can engage with an exemplary trocar that is configured to have one or more stabilization mechanisms on a shaft thereof such that the trocar provides a stable delivery mechanism to the injection device. In certain embodiments, the trocar is configured to receive an injection needle therein that is attached to the injection device, and the injection device can deliver a dose as a continuous flow of fluid therethrough to a target tissue site. In certain embodiments, the trocar is configured to receive an injection needle therein that is attached to the injection device, and the injection device can deliver fluid therethrough in bolus doses to a target tissue site. In certain embodiments, the devices, systems, and methods provided herein can be used in a variety of treatments, such as providing measured dosing of therapeutic cells or cell products to a kidney (e.g., in a patient who has kidney disease such as chronic kidney disease) by direct delivery of fluid containing therapeutic cells or cell products to multiple extravascular injection sites in the renal parenchymal/stromal compartments of a patient. In certain embodiments, a patient is positioned in a prone or lateral position, as illustrated in FIGS. 1 and 2. The “parenchyma” is the functional tissue of an organ as distinguished from the connective and supporting tissue. The “stroma” is the supportive tissue of an epithelial organ, tumor, gonad, etc., consisting of connective tissues and blood vessels. In certain embodiments, a subject has late-stage failure. However, while the disclosure discusses treating kidneys herein, the devices, systems, and methods can be utilized broadly. In certain embodiments, the devices, systems, and methods provided herein can be used in a variety of treatments, such as providing measured dosing of therapeutic cells or cell products to a tissue site that has cancer cells, such as a solid tumor. In certain embodiments, the tissue site is an organ that has or is suspected of having cancer cells (e.g., metastatic cancer cells or a tumor).

In certain embodiments, the devices, systems, and methods provided herein are useful for the percutaneously injection of pharmaceutical fluid formulations into a cancer-cell or solid tumor containing organ and/or directly into a tumor. In certain embodiments, it is important to distribute the formulation in the organ as widely as possible. In certain embodiments, distributing the formulation in the organ or tumor is achieved by entering the organ or tumor at an angle allowing deposition of the formulation in the organ or tumor as widely as feasible. In certain embodiments, the organ or tumor is imaged in a longitudinal or transverse approach using ultrasound guidance or with axial computed tomography (CT) imaging, depending upon individual patient characteristics. In certain embodiments, the injection involves multiple deposits as the injection needle is gradually withdrawn. In certain embodiments, the full volume of the formulation may be deposited at a single or multiple entry points. In certain embodiments, up to two entry points may be used to deposit the full volume of therapeutic formulation into the organ or tumor.

In certain embodiments, the devices, systems, and methods provided herein are useful for the percutaneously injection of pharmaceutical fluid formulations into the renal cortex of a kidney. In certain embodiments, it is important to distribute the formulation in the renal cortex as widely as possible. In certain embodiments, distributing the formulation in the renal cortex is achieved by entering the renal cortex at an angle allowing deposition of the formulation in the renal cortex as widely as feasible. In certain embodiments, the kidney is imaged in a longitudinal or transverse approach using ultrasound guidance or with axial CT imaging, depending upon individual patient characteristics. In certain embodiments, the injection involves multiple deposits as the injection needle is gradually withdrawn. In certain embodiments, the full volume of the formulation may be deposited at a single or multiple entry points. In certain embodiments, up to two entry points may be used to deposit the full volume of therapeutic formulation into the kidney. In certain embodiments, the injection may be administered to a single kidney, using one or more entry points, e.g. one or two entry points. In certain embodiments, the injection is made into both kidneys, in each kidney using one or more entry point, e.g. one or two entry points.

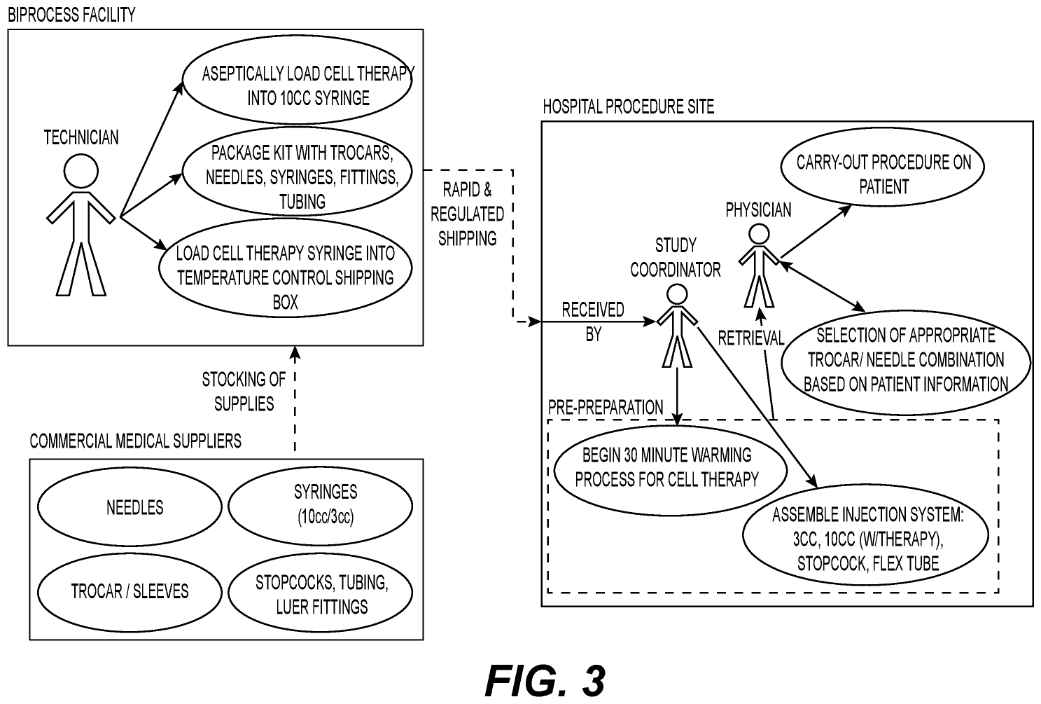

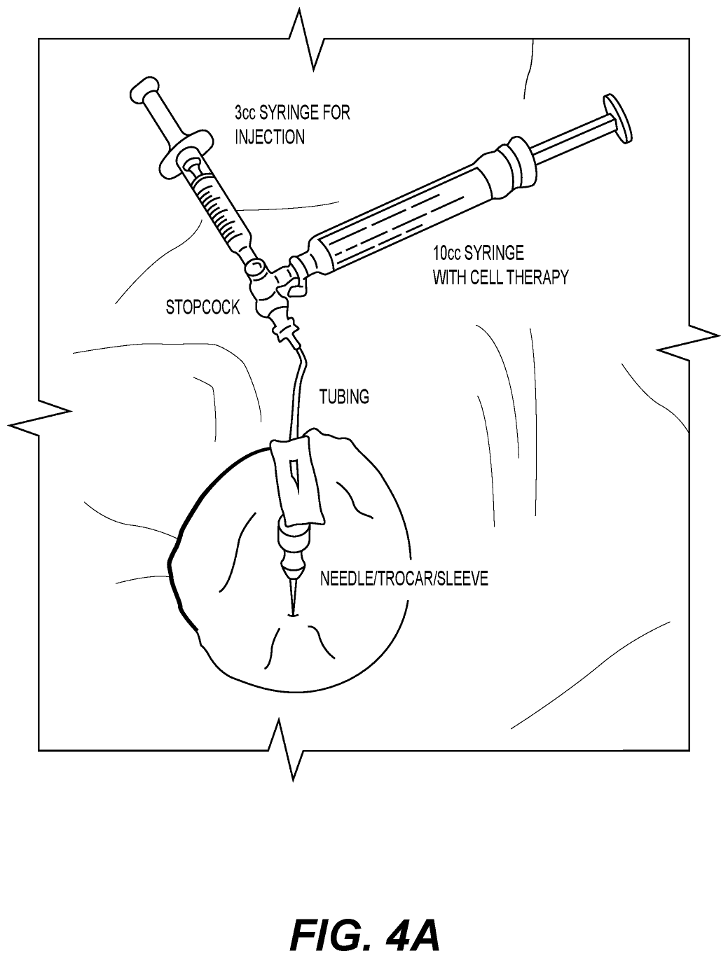

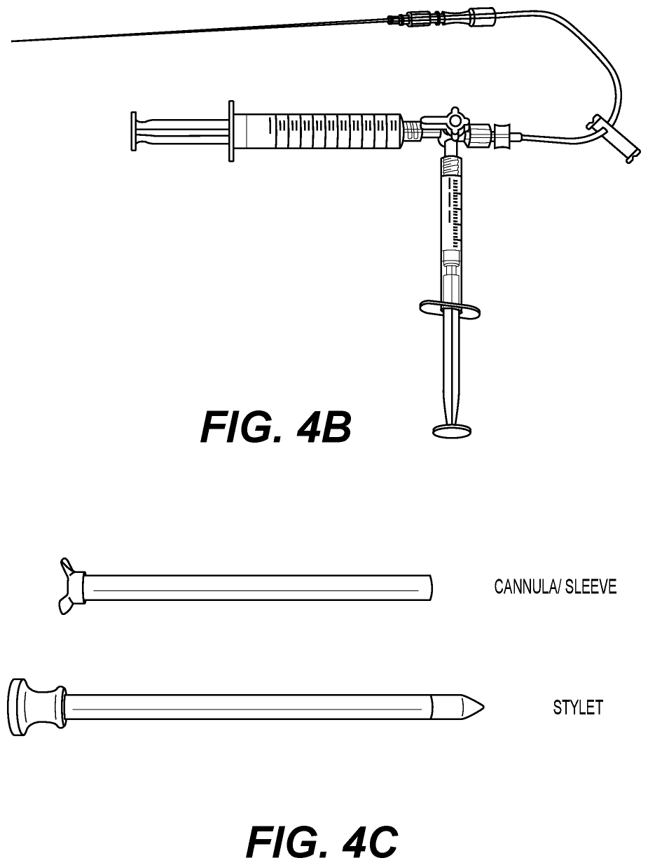

The devices and systems provided herein provide advantages over commercially available devices. FIGS. 3 and 4 outline a process for performing cell therapy using off-the-shelf components on a kidney of a patient. For example, off-the-shelf parts such as needles, trocar/sleeves, luer fittings, stopcocks, tubing, 3 cc syringes, and 10 cc syringes can be stocked at a site where therapeutic cellular material is processed. A kit can be prepared that contains the aforementioned items in replicate, with a size range for various needles and trocar/sleeves. A range of gage sizes for the needles can be provided with a range of lengths: 10 cm, 15 cm, and 20 cm. A 10 cc syringe that can be used in a package that allows aseptic filling with the cell therapy can be loaded. The loaded syringe can then shipped separately to the site of the procedure in a temperature control package that maintains the therapy at about 4 to 8° C. during the course of transit. At the procedure site, a protocol can be implemented to warm the syringe, assemble the injection system, and organize and prepare the supplies for the user. For example, the syringe can be warmed to about 26 to 28° C. over a controlled period of time, such as over about 30 minutes. The user can then begin the procedure within the allotted window for product viability, which can be about 1.5 hours. FIG. 4 illustrates the non-limiting injection system as discussed above in which the 3 cc syringe is connected to the 10 cc syringe via a 3-way stopcock. The output from the stopcock is sent via tubing to a luer connection on the injection needle within the trocar/sleeve placed in the patient. The cell therapy can initially move into the 3 cc syringe via the 10 cc plunger, and it can be subsequently injected into the kidney of the patient with the control and ease of plunger movement provided by the smaller syringe. Injecting the cell therapy can be a challenging endeavor that can require at a minimum both hands of the lead user, and often another hand in assistance to work the plunger while stabilizing the trocar, the needle, and the injection system. The devices and systems included herein provide advantages over the processes and components illustrated in FIGS. 3 and 4, such as improved stability (and reduced damage) during delivery of a therapeutic agent, as well as more consistent delivery of the therapeutic agent (e.g., amount and location).

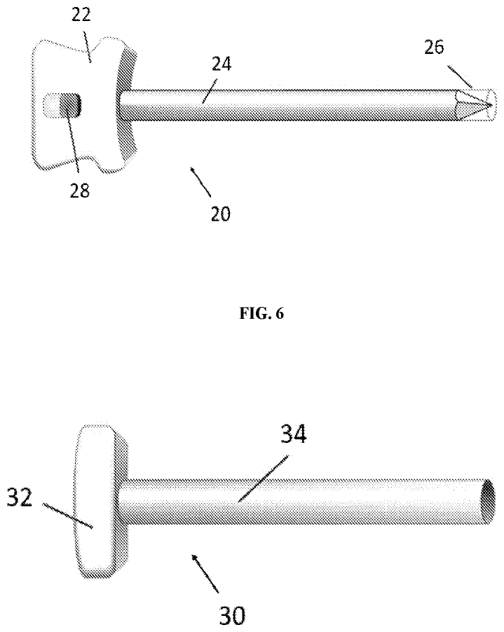

FIG. 5 illustrates one non-limiting embodiment of a trocar 20 that can be used herein. As a non-limiting example, in its simplest form, a trocar can be an approximately pen-shaped instrument with an at least somewhat sharp triangular point at one end, often used inside a hollow tube known as a cannula or sleeve, to create an opening into the body through which the sleeve may be introduced, to provide an access port during surgery. In certain embodiments, the trocar is a pen-shaped instrument with two parts, a solid obturator/stylet with a sharp triangular point at one end inside of a hollow tube, known as a cannula or sleeve (e.g., the trocar is used to create an opening into the body: the stylet may be removed, leaving behind the sleeve to provide an access port to internal structures). The trocar 20 as illustrated in FIG. 5 can have a handle 22, an elongate shaft 24, a shield 26, and a shield release 28. A lumen can extend therethrough. FIG. 6 illustrates one non-limiting of a cannula 30 that can be used herein. The cannula 30 has a handle 32 and a sleeve 34, and it can have a lumen extending therethrough. When a trocar, such as trocar 20, is placed in a patient, fixation of the trocar is largely accomplished at two points. Frictional gripping of the trocar occurs at the skin-pass-through, and similar, weaker interaction occurs on the trocar by internal tissue, such as the capsule of the kidney. The frictional gripping at the internal tissue is weaker because of the shallow penetration depth of the trocar. However, stabilizing the penetration depth of the trocar into tissue such as the kidney during the procedure can be beneficial to avoid trauma to tissue and to assist in a smoother injection of therapeutic cellular material, and detrimental movement of the dermal anchor point in relation to the inner tissue anchor point, such as at the kidney, can occur.

### Injection Device

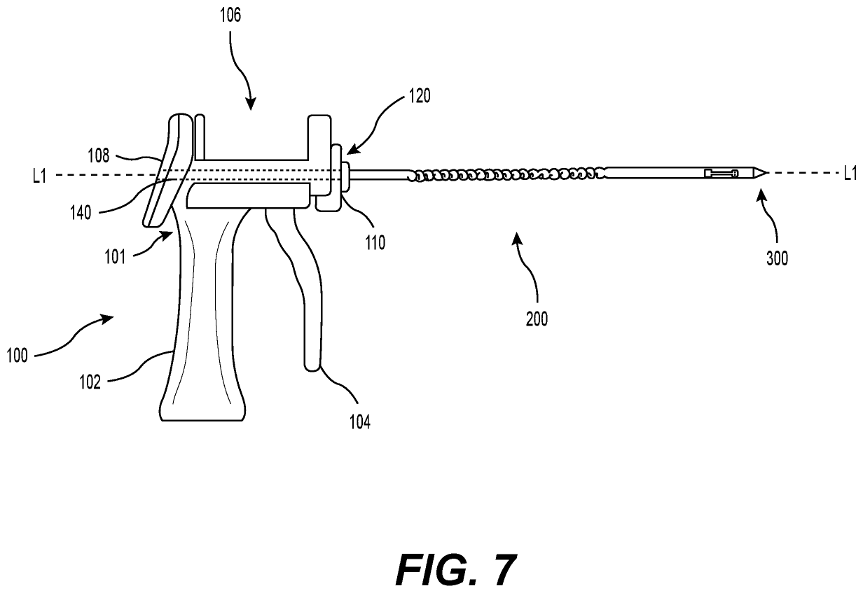

FIG. 7 illustrates one non-limiting example of an injection device 100 that can be configured for one-handed operation with a detachable trocar 200 that has a stylet 300 therein and that extends distally from the device 100 along a longitudinal axis LI of the device 100. The injection device 100 has a housing 101, a handle 102, an actuator 104, a fluid receiver 106, a display 108, and a trocar engagement mechanism 110. In certain embodiments, the display 108 is omitted. In certain embodiments, within the device 100, there is a pump (not shown), one or more sensors configured to detect a variety of conditions, such as pressure, flow rate, temperature, etc. (not shown), a power source such as a battery (not shown), and/or a central processing unit (CPU) (not shown). In FIG. 7, the handle 102 extends from the housing 101 and is in the shape of a pistol grip. However, a variety of handles, grips, controls, etc. can be used.

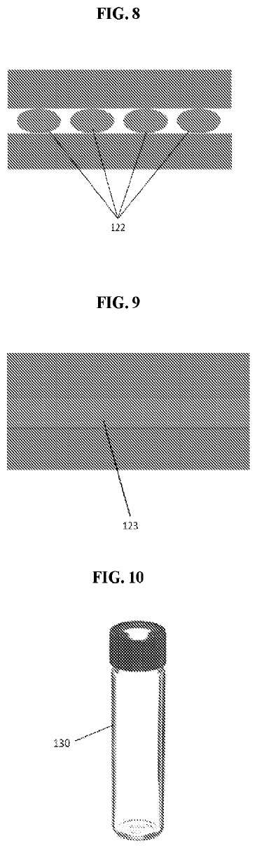

The actuator 104 is a trigger that is configured to actuate delivery of a fluid from the fluid receiver 106 and through a valve 120 on a distal end of the injection device 100, discussed in more detail below. While the actuator 104 is illustrated as a trigger, the actuator can have various forms, such as plungers, buttons, switches, electronic actuations, CPU-actuated means, incorporated into the display 108, etc. The actuator 104 is configured to be manually depressed towards the handle 102. In certain embodiments, as the actuator 104 is depressed, the actuator 104 can be configured to provide haptic feedback to a user. In certain embodiments, the haptic feedback can be physically created from manually depressing the actuator 104 or it can be simulated. For example, the device 100 can have one or more haptic feedback mechanisms built therein that are configured to simulate a mechanistic action of the actuator 104 that is in fact electromechanical and controlled by the CPU. In certain embodiments, depression of the actuator 104 is configured to deliver fluid from the fluid receiver 106 through the valve 120 in a pulse-delivery pattern that includes delivering discrete boluses 122 of fluid to a target site, as illustrated in FIG. 8, and it is configured to retract an injection needle from the target site during fluid delivery to allow expansion space for the delivered fluid. In certain embodiments, the discrete boluses 122 of fluid can be configured not to be in direct contact with each other, which can prevent or reduce extravasation from a target tissue site. In certain embodiments, the device alternatively can be configured to deliver continuous streams 123 of fluid as illustrated in FIG. 9 and/or a combination of the two. In certain embodiments, the pulse-delivery pattern and needle retraction can be created by an electromechanical system with a pumping mechanism within the device 100, which can be created by the pump and CPU. However, the pumping and retraction mechanism can also be mechanical in nature, or the entire device 100 can be purely mechanical. In certain embodiments, the fluid can be drawn from a fluid reservoir engaged with the fluid receiver 106.

In certain embodiments, as shown in FIG. 7, the fluid receiver 106 is positioned on an upper surface of the device 100. However, it can be incorporated anywhere on the device 100. In certain embodiments, the fluid receiver 106 is configured to receive fluid from the fluid reservoir, such as a cartridge 130 shown in FIG. 10 or a syringe, and configured to deliver fluid to the valve 120 upon actuation of the actuator 104. In certain embodiments, the fluid receiver 106 is configured to receive the cartridge 130 at least partially therein and pierce the cartridge 130 using an aseptic septum-piercing element therein. However, the fluid receiver 106 can alternatively be configured to receive one or more cartridges 130 entirely therein, connect to one or more fluid lines, etc. The fluid receiver 106 can also alternatively have one or more valves, fittings, engagements, etc. therein to connect to fluid reservoir(s). In certain embodiments, the fluid receiver 106 can also have temperature control built into the housing 101 to control a temperature of the cartridge 130. In certain embodiments, the cartridge 130 comprises a hydrogel (e.g., a gelatin-based hydrogel) that is heated until the hydrogel melts into a liquid. In certain embodiments, a therapeutic agent (e.g., a cell population or cell product such as an exosome) is dispersed (e.g., uniformly) throughout the hydrogel and/or fluid.

The fluid reservoir, such as the cartridge 130, is configured to deliver one or more fluids through the device 100 and to a target site upon actuation of the actuator 104. The cartridge 130 illustrated in FIG. 10 is a glass vial cartridge with a rubber diaphragm interface that is configured to deliver fluid upon being pierced by the aseptic septum-piercing element of the fluid receiver 106. In certain embodiments, the cartridge comprises material characteristics that take into account viscosity of a therapeutic agent (such as cell therapy) thus preventing lost therapeutic agent adhering on the inner walls of the cartridge. In certain embodiments, the cartridge 130 can be assembled with the device 100 in a sterile field, and the aspiration pathway that enters the cartridge to connect to a fluid pathway through the device 100 can be configured to prevent loss of the fluid due to access in various use case orientations of the cartridge 130, for example if the cartridge 130 is inverted. The cartridge 130 can also be configured to be securely retained by the device 100 in the fluid receiver 106. In certain embodiments, the fluid reservoir can be made from a variety of materials, such as polymers, rubber, etc. and can have a variety of forms, such as pouches, fluid lines, containers, etc. that can connect with the fluid reservoir 106 in a variety of means, such as through ports, valves, etc. The cartridges 130 can also configured to be removable and replaceable, for example after delivering the fluid therein. In certain embodiments, the cartridges 130 can be provided in preselected doses and/or configurations such that users can use a plurality of cartridges 130 during one treatment depending on the desired treatment. Alternatively, the device 100 can have a built-in fluid reservoir that is intended for a single use. In certain embodiments, the device 100 can also have one or more mechanisms for setting a customized dose from a cartridge 130, such as through use of the display 108 (discussed below). In certain embodiments, the cartridges 130 can have one or more computer chips therein that connect with the device 100 upon insertion and provide details regarding the contents of the cartridge 130, recommended doses, flow rates, timing, etc. The computer chips can connect with the CPU of the device 100 through one or more means, such as through wired connections disposed in the fluid receiver 106 and/or wirelessly.

In certain embodiments, the cartridge 130 can be configured to connect with the device 100 such that minimum dead volume is generated upon connection, and the cartridge 130 can be configured to be aseptically filled at the manufacturing source with fluid as needed for cell therapy and transported to a surgery site while maintaining a transport temperature of from about 0° C. to about 20° ° C., and more particularly about 2° C. to about 8° C. or about 4° C. to about 8° C. In certain embodiments, the device 100 can be configured to receive the cartridge 130 that can have a lowered transportation temperature, as detailed above, to warm the cartridge 130 to a temperature for delivery to a patient within a selectable time period, and to maintain the cartridge 130 at a temperature that is acceptable for use for another selectable time period and/or until the cartridge 130 is used. For example, the device 100 can be configured to receive a cartridge 130 whose contents are at a transportation temperature, such as about 4° C. to about 8° C. The device 100 can then warm the cartridge 130 to a temperature for use in a patient, such as from about 20° ° C. to about 40° C., and more particularly about 25° C. to about 37° C., within a certain time period, such as within 15 minutes, within 30 minutes, or within 45 minutes. The device 100 can then be configured to hold the temperature of the cartridge 130 about constant until the cartridge 130 is used or for a fixed time period, for example about 1.5 hours. In some non-limiting examples, the device 100 can be configured to indicate to a user if the cartridge 130 has been heated incorrectly and/or if the cartridge 130 has not been used within the allotted window for sample viability. In certain embodiments, the device 100 can prevent the user from using the cartridge 130 if the device 100 determines sample viability is unacceptable, such as preventing actuation of the device 100. The volume of the cartridge 130 can vary depending on the desired treatment. For example, the range of volumes used for cell therapy can be from about 1 ml to about 15 ml, or more particularly from about 3 ml to about 8 ml. In use during renal treatment, the volumes of the cartridges 130 used can depend on patients' renal mass. Table 1 illustrates exemplary dosage volumes that can optionally be used herein when treating a kidney.

TABLE 1

| | |
|---|---|
| Estimated Kidney Weight (g KWₑₛₜ)* | Dosing Volume |

| Median Weight (g) | Weight Range (g) | (mL) |
|---|---|---|
| 100 | 95-108 | 3.0 |
| 117 | 109-125 | 3.5 |
| 133 | 126-141 | 4.0 |
| 150 | 142-158 | 4.5 |
| 167 | 159-175 | 5.0 |
| 183 | 176-191 | 5.5 |
| 200 | 192-208 | 6.0 |
| 217 | 209-225 | 6.5 |
| 233 | 226-241 | 7.0 |
| 250 | 242-258 | 7.5 |
| — | >259 | 8.0 |

The pharmaceutical fluid formulation can include a variety of therapeutic treatments, such as therapeutic cells and/or a products thereof such as exosomes) suspended in liquid. For example, use in a kidney can utilize fluid including therapeutic cells and a supporting hydrogel with a viscosity of, for instance, about 1.05 to 1.35 cP. The viscosity of the fluid can introduce further considerations when using the components disclosed herein, such as an injection needle and a syringe with a barrel and plunger. For example, to initiate flow from a component such as the injection needle, an application of force is required to the plunger of the syringe that is greater than the force required to maintain the flow when it has started. Additionally, there can be an initial surge of fluid, such as the therapeutic cells and supporting hydrogel, from the needle that is beyond manual control of the syringe plunger. Once the flow starts, removal of the plunger force may not stop the flow of fluid. There can be hysteresis in the pressure build-up in the barrel of the syringe, and the outflow of fluid may only be stopped by retracting the plunger. Because of these considerations, users may act to pulse the plunger in use in order to maintain better control of the flow from the needle.

In certain embodiments, the viscous nature of a therapeutic agent (such as cell therapy, e.g., a pharmaceutical fluid formulation comprising cells) might tend to create a situation where some of the therapeutic agent is lost—that is, the therapeutic agent (e.g., a portion thereof) adheres to the inside of the cartridge and cannot be removed through the normal process of the device. In certain embodiments, the inside surface of the cartridge is hydrophobic. In certain embodiments, the inside surface of the cartridge is superhydrophic. In certain embodiments, the inside surface of the cartridge is hydrophobic or superhydrophic to reduce wetting and adherence of the therapeutic agent (e.g., cell therapy) to the cartridge. In certain embodiments, the inside surface of the cartridge is hydrophobic or superhydrophic to prevent the therapeutic agent (e.g., cell therapy) from wetting and adhering to the cartridge.

A variety of fluids can be used herein, such as those discussed in U.S. Pat. No. 8,318,484 issued Nov. 27, 2012: PCT International Publication No. WO/2011/143499 published Nov. 17, 2011: U.S. Pat. No. 9,724,367 issued Aug. 8, 2017: U.S. Patent Application Publication No. 2017-0281684 published Oct. 5, 2017; and U.S. Patent Application Publication No. 2016-0101133 published Apr. 14, 2016, which are all incorporated herein by reference in their entirety. The fluid receiver 106 is thus configured to fluidly connect the cartridge 130 with the valve 120 to allow the fluid to be delivered therethrough.

In the exemplary device illustrated in FIG. 7, the valve 120 is positioned internally at a distal end of the device 100 where the trocar 200 connects to the device 100. In certain embodiments, the valve 120 is a luer hub that is configured to connect with an injection needle for delivery of fluid therethrough. However, a variety of valves configured to connect to an injection needle can be used. In certain embodiments, the valve 120 engages with the housing 101 of the device 100 through a flexible mounting that is configured to translate longitudinally distally and proximally along the axis LI of the device 100 that is approximately parallel to a fluid flow path through the injection needle. In certain embodiments, the valve 120 can translate distally and proximally up to about 5 cm (e.g., up to about 1, 2, 3, 4, or 5 cm), and more particularly up to about 2 cm. Thus the valve 120 is configured to translate distally and proximally upon actuation of the device 100 to dispense fluid therethrough, allowing steadier delivery of fluid to a target delivery site and proximal retraction of an injection needle from the target site during fluid delivery.

In certain embodiments, a display 108, such as an I/O touch screen, is positioned on a proximal end of the device 100, such as shown in FIG. 7. In certain embodiments, the display 108 is configured to interact with the CPU to allow a user to control various functions and features on the device 100. For example, the display 108 can allow the user to set injection parameters, prime an injection needle, monitor various pressure levels and fluids delivered, remove interlocks between components, provide real-time dosing information, etc. In an example, the display 108 is a touch screen with a plurality of input controls displayed thereon. However, the display 108 can alternatively and/or additionally have one or more physical buttons, controls, switches, dials, gauges, toggles, etc. for controlling one or more of the functions of the device 100. The display 108 can also be positioned anywhere on the device 100, such as on a top or sides of the device 100.

In certain embodiments of the device 100, the housing 101 can also have a stylet removal lumen 140 that extends along the axis LI and that is configured to allow removal of the stylet 300 therethrough after placement of the trocar 200 (discussed below). The lumen 140 can allow manual removal of the stylet 300, for example being configured to allow the stylet 300 to extend proximally from a proximal end of the lumen 140 to allow manual grasping, or the lumen 140 can incorporate one or more mechanical and/or electrical mechanisms to provide removal of the stylet 300, for example by using one or more gears, wheels, hooks, moving tracks, etc. In certain embodiments of the injection device, needle penetration depths can be variably set on the device itself.

The device 100 can have a variety of sizes as needed, for example it can fit within a space of about 300 mm width by about 200 mm depth by about 100 mm height, and more particularly it can fit within a space of about 205 mm width by about 105 mm depth by about 70 mm height. The device 100 can have a variety of weights and/or masses as needed. In certain embodiments, the device 100 can have a mass of less than about 2000 g, and more particularly a mass of less than about 1400 g. The device 100 can be made from a variety of materials, such as metal, resin, etc. or a combination of materials. The device 100 can be configured to be single-use or can be configured to be a reusable device that requires resterilization. In certain embodiments, cell-contacting materials are configured to meet certain use requirements, such as ISO10993, to address issues such as risk of leachables and compatibility with sterilization (over multiple cycles). Descriptions relating to ISO10993 are provided in Guidance for Industry and Food and Drug Administration Staff (Document issued on: Jun. 16, 2016) entitled: Use of International Standard ISO 10993-1, “Biological evaluation of medical devices—Part 1: Evaluation and testing within a risk management process”, U.S. Department of Health and Human Services Food and Drug Administration Center for Devices and Radiological Health (available at www.fda.gov/downloads/medicaldevices/deviceregulationandguidance/guidancedocuments/ucm348890.pdf), the entire contents of which are incorporated herein by reference. The materials used for the device 100 can also be consistent with regulations for Class I/II devices, and the device 100 can optionally avoid using lubricants to avoid impacting viability of the therapeutic cellular material. Descriptions relating to Class I/II devices are provided in Medical Devices “Classify Your Medical Device” as updated on Mar. 27, 2018 U.S. Food & Drug Administration (available from www.fda.gov/MedicalDevices/DeviceRegulationandGuidance/Overview/Classify YourDevice/ucm2005371.htm), the entire contents of which are incorporated herein by reference. In various embodiments, the device 100 can have an interlock to prevent accidental deployment of any loaded cartridge 130 or therapeutic cellular material. Additionally, in other non-limiting embodiments that incorporate a dynamic injection system to assist the penetration of tissue, such as the kidney capsule, an interlock system can be used to prevent accidental triggering when the device is not in proper position with respect to tissue. In some embodiments, the device 100 can require a power source, and the power source can be rechargeable. For example, the device can incorporate a charging interface, such as a USB interface. In certain embodiments, button interfaces will likely need to comply with usability standards found in IEC 62366. See, e.g., the International Electrotechnical Commission (2014). Application of usability engineering to medical devices, International IEC Standard 62366 edition 1.1 2014-01. International Electrotechnical Commission, entire contents of which are incorporated herein by reference.

The device 100 can couple to the detachable trocar 200 through the engagement mechanism 110, which can include a variety of different friction fit openings, snaps, hooks, levers, etc.

Trocar with Stylet

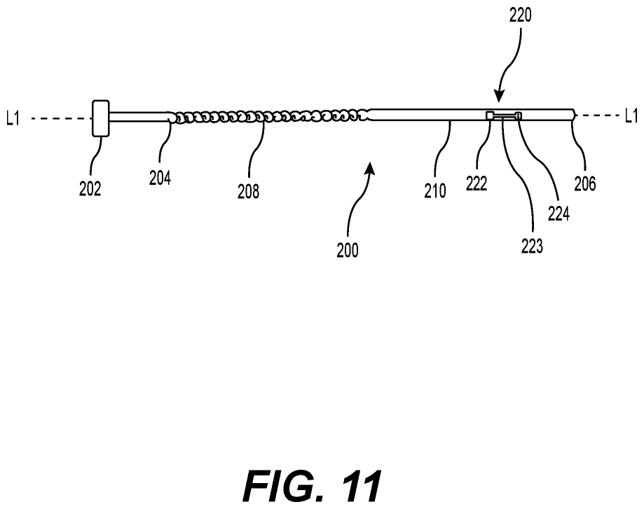

FIG. 11 illustrates one non-limiting example of a trocar (which may be, e.g., detachable to a device as provided herein). The trocar 200 is configured to be placed within a patient and provide access to an internal tissue site, such as a kidney tumor-containing organ, or tumor. The trocar 200 of FIG. 11 has a flared head 202 and a hollow elongate cylindrical shaft 204 extending distally therefrom. The head 202 is configured to be removably and replaceably attached to the device 100 along the axis LI at the engagement mechanism 110. A lumen extends through the head 202 and the elongate shaft 204, and it is configured to receive instruments, such as the stylet 300 and an injection needle, therethrough. The elongate shaft 204 has a tapered distal end 206 and a stabilization mechanism 220 on a distal portion thereof. The shaft 204 has a compressive spring section 208 and a solid section 210. The compressive spring section 208 is configured to compress and expand with movement of a patient and interaction with the device 100. The spring section 208 thus allows translational motion proximally and distally along the axis LI, allowing more stationary interaction with tissue, smoother delivery of fluid to a target tissue site, and proximal retraction of an injection needle from the target site during fluid delivery. In certain embodiments, the spring section 208 can allow translation distally and proximally up to about 5 cm (e.g., up to about 1, 2, 3, 4, or 5 cm), and more particularly up to about 2 cm. The compressive spring section 208 is illustrated at a middle portion on the shaft 204. However, the compressive spring section 208 can be positioned at various points along the shaft 204, for example at a position proximal to a midpoint of the shaft 204 or a position distal to the midpoint. The solid section 210 is positioned on a distal portion of the shaft 204, and it has the stabilization mechanism 220 thereon.

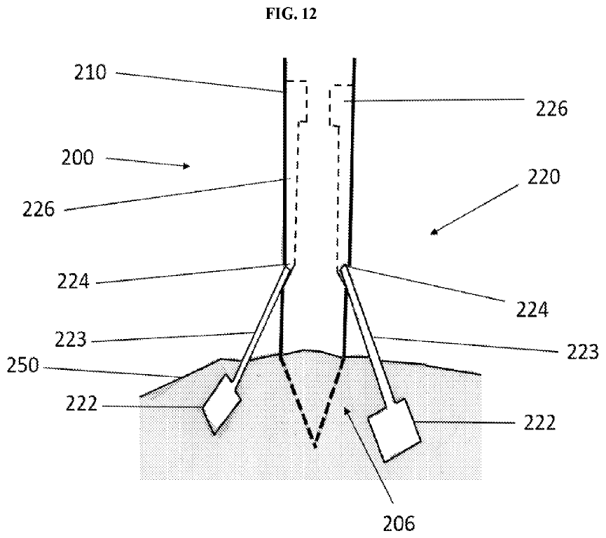

The stabilization mechanism 220 is configured to help stabilize the trocar 200 with respect to an interior tissue site within a patient when the trocar 200 has been placed through an outer tissue surface, such as a dermis, of the patient. The stabilization mechanism 220 is thus configured to reversibly keep the trocar 200 at a fixed position relative to tissue as instruments are passed through the trocar 200. For example, when the trocar 200 is used during treatment of a kidney, the stabilization mechanism 220 can be configured to reversibly lock the distal end 206 of the trocar 200 in position relative to a surface 250 of the kidney. The illustrated stabilization mechanism 220 in FIGS. 11 and 12 includes three feet 222 on shafts 223 engaged with the shaft 204 at hinged points 224. In certain embodiments, the feet 222 are radially symmetrically-located around the shaft 204 and are configured to deploy to reversibly engage tissue at a target site. In certain embodiments, the feet 222 are configured to move from a recessed position in the shaft 204, for example during insertion of the trocar 200 into a patient, to an expanded position, for example during stabilization of the trocar 200 in use, by rotating away from the shaft 204 about the hinged points 224 to an engaged position, as illustrated in FIG. 12. In the recessed position, the feet 222 can be received in recession pockets 226 such that the feet 222 sit flush with an outer surface of the shaft 204. During deployment, the feet 222 can be configured to rotate away from the shaft 204 by between 90 and 180 degrees, for example by about 100 degrees, to engage with tissue.

A variety of rotation mechanisms can be incorporated into the shaft 204 to cause rotation of the feet 222, including gears, springs, electronic motors, shafts, electrically heated nitinol struts, etc. For example, small cams (not shown) can be incorporated into the shaft 204 approximately at the hinged points 224 that can be configured to engage one or more features on the stylet 300 upon removal of the stylet 300, which removal can be configured to actuate the cams and cause rotation of the feet 222. The stabilization mechanism 220 generally can have one or more deployment means that are actuated upon removal of the stylet 300. In certain embodiments, the feet 222 can have one or more engagement means thereon, for example biomimetic micro-hooks configured to reversibly engage tissue upon initial surface contact. The micro-hooks can have a variety of sizes, such as being from about 100 to 500 μm in scale. The trocar 200 is thus secured in place without causing significant trauma until a user wants to extract the trocar 200 the feet 222 are released. A variety of other engagement means are possible, for example biomimetic or other hooks, temporary adhesives, active suction, friction, spring-force, a mechanism similar to a lamprey's latch mechanism, etc. The engagement means can also be sacrificial and/or biodegradable. While three feet 222 are illustrated, a plurality of feet can be provided. Additionally, the stabilization mechanism 220 can have various alternative and/or additional embodiments other than feet 222. For example, the stabilization mechanism can include pincer mechanisms, hooks, various adhesives and glues, suction, friction fit, etc.

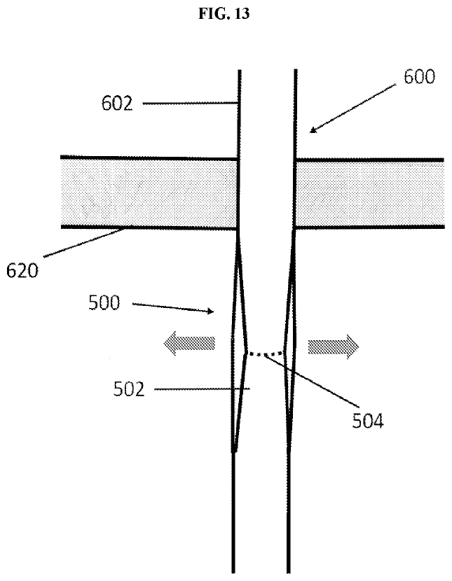

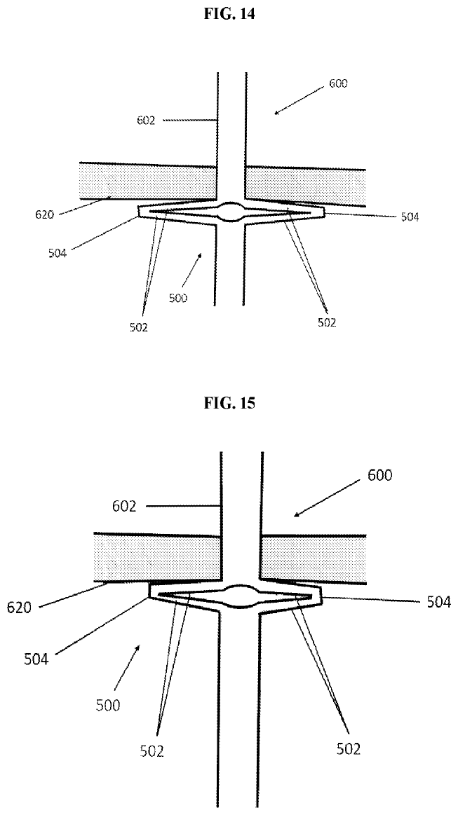

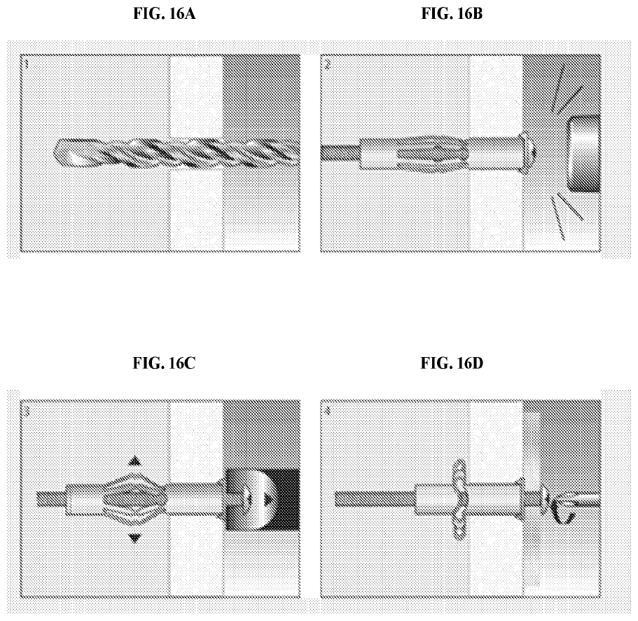

The trocar 200 can also have one or more additional or alternative engagement mechanisms for engaging tissue and further stabilizing the trocar 200 when placed in a patient, such as at an entry point of the trocar 200 in a dermis of the patient. For example, the trocar 200 as illustrated is configured to be frictionally gripped by the dermis of the patient when placed. However, additional engagement mechanisms are possible, such as a reversibly collapsible, expanding element 500 that can be incorporated into a shaft 602 of a trocar 600 similar to the trocar 200. In certain embodiments as illustrated in FIGS. 13-15, the expanding element 500 can operate similar to a drywall anchor (such as an exemplary drywall anchoring system as illustrated in FIGS. 16A-16D outlining drilling a hole, hammering in an illustrative drywall anchor, using a screwdriver to expand anchoring arms, and finally screwing a screw into place). The expanding element 500 is configured to move between a placement position, as illustrated in FIG. 13 in which the shaft 602 is inserted through a dermis 620 of a patient, to an expanded position, as illustrated in FIGS. 14 and 15. The expanding element 500 has one or more legs 502 that are hinged or bendable at an approximate midpoint 504 along the legs 502 (illustrated in FIG. 13 by a broken line). In certain embodiments, one or more expansion mechanisms can be incorporated into the shaft 602 and configured to cause the legs 502 to expand outwards, such as threading configured to retract a distal portion of the trocar 600 proximally after it is positioned within the dermis 620 such that the legs 502 expand outwards (as shown by arrows in FIG. 13) to secure against an inner surface of the dermis 620 (as illustrated in FIGS. 14 and 15). The expanding element 500 is configured to be reversed to the placement position by a user when the user wants to remove the trocar 600 from the patient.

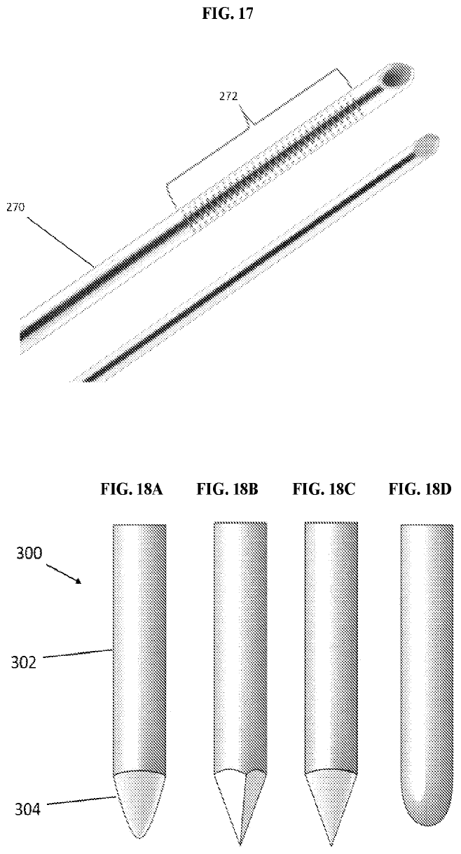

The exemplary trocar 200 is thus configured to be stabilized and/or fixed at two points when the trocar 200 is placed in a patient, at a surface of the interior target tissue (such as an organ such as a kidney or a solid tumor) and at an entry point through tissue into the patient (such as in the dermis) using one or more of the mechanisms discussed above. Stabilization of the trocar 200 is configured to allow a stable penetration depth in tissue during deployment, which prevents or reduces damage to the tissue and reduction of loss of excess therapeutic cells or cell products during injection from shifting instruments caused by shifting tissue and movement of the patient, such as natural movement of an organ and respiration of the patient. In certain embodiments, one or more surface features can also be added to an outer distal surface of the shaft 204 of the trocar 200, such as bumps, grooves, holes, markings, etc., that are configured to provide better visualization of a location of the trocar 200, such as under ultrasound, and thus more accurate placement within a patient. For the sake of illustration, exemplary echogenic surface features 272 are illustrated on a non-limiting embodiment of an ultrasound biopsy needle in FIG. 17. Echogenic can be considered to mean possessing the property of being visible to ultrasound imaging. As a non-limiting example, the illustrated needle is a Cook Medical EchoTip®. The trocar 200 can optionally be used with a cannula or trocar sleeve as desired, such as a trocar sleeve that is at least about 20 g large, and it can be sized and shaped to penetrate a patient to a variety of depths, such as about 3 to 5 mm into an internal target tissue site like the patient's kidney. The trocar 200 can have a variety of lengths, such as from about 5 to 25 cm, more particularly from 10 cm to 20 cm, and even more particularly from about 15 to 20 cm. The spring section 208 can have a variety of lengths, such as from about 5 to 10 cm.

The stylet 300 is configured to be received within the lumen of the trocar 200 along the axis LI during advancement and placement of the trocar 200 within a patient. The stylet 300 has an elongate stylet shaft 302 with a distal tip 304. The shaft 302 is sized and shaped to be received within the trocar 200 and extend both distally and proximally from the trocar 200. For example, the distal tip 304 can be configured to extend distally from the tapered distal end 206 of the trocar 200 such that the distal tip 304 can pierce tissue, and a proximal end of the stylet 300 can extend proximally from the trocar 200 for removal of the stylet 300. An exemplary distal tip 304 has a blunt conical shape as illustrated in FIG. 18A, but a variety of other shapes can be used, such as pyramidal as illustrated in FIG. 18B, sharp conical as illustrated in FIG. 18C, and blunt as illustrated in FIG. 18D. While a variety of tips can be used, in certain embodiments, the blunt conical tip can be used effectively in kidney tissue because the blunt conical tip is configured to minimize trauma caused when initially inserting the trocar 200 with the stylet 300 into a target tissue site, such as the kidney. After placement of the trocar 200 within a patient, the stylet 300 can be configured to be removed. The stylet 300 can be configured to be manually removed from the trocar 200 directly when the device 100 is not engaged with the trocar 200, or it can be configured to be removed from the trocar 200 through the optional removal lumen 140 of the device 100, either manually, mechanically, electrically, or some combination, as discussed above. In certain embodiments, the stylet 300 can have one or more features configured to actuate deployment of the stabilization mechanism 220 of the trocar 200 upon removal of the stylet 300 therefrom. For example, the stylet 300 can have one or more gear-tooth features positioned towards the distal tip 304 that are configured to actuate the cams on the trocar 200 and cause rotation of the feet 222.

Thus in a non-limiting example, the trocar 200 (with or without a sleeve) can be configured to pierce a skin of a patient through action of the stylet 300. An exemplary target penetration depth of the trocar 200 and/or sleeve into tissue, such as an organ (e.g., a kidney), can be about 2 to 6 mm, or more preferably about 3 to 5 mm when the stylet 300 is removed. When the stylet 300 is removed, the trocar 200 (i.e., the sleeve of the trocar) can then be fixed in place. Fixation of the trocar 200 (i.e., the sleeve of the trocar) can be accomplished at least at two points, frictional gripping at the dermis passage and similar, weaker interaction in the tissue into which the trocar (i.e., the sleeve of the trocar) 200 penetrates, such as the capsule of the kidney. The trocar (i.e., the sleeve of the trocar) 200 can be configured to be stabilized at the penetration depth into the tissue, such as an organ (e.g., a kidney), during the procedure. However, placement of components penetrating the issue can be dynamic due to potential shifting of tissue, such as an organ (e.g., a kidney), and through respiration cycle of the patient. Therefore, detrimental movement of the dermal anchor point in relation to the anchor point of the internal tissue, such as an organ (e.g., a kidney), can occur and can thus be minimized using the components provided herein, such as the stabilization mechanism 220. Minimizing trauma to tissue, such as an organ (e.g., a kidney), can also be beneficial to overall success of any treatment. For example, trauma can originate when there is unintended movement of the needle 400 in the trocar 200 that lacerates the tissue, such as an organ (e.g., a kidney). Trauma can also occur when initially inserting the trocar 200 into tissue, such as an organ (e.g., a kidney), for example if the distal tip 304 of the stylet 300 has any cutting action (such as a sharp point as occurs in the pyramidal or sharp conical designs discussed above). Thus stabilization of the trocar 200 can help reduce or eliminate trauma to the tissue by reducing or eliminating unintended movement of the needle 400, and the blunt conical tip can be used for piercing tissue, such as an organ (e.g., a kidney), and minimizing trauma.

In certain embodiments, trauma can originate from two sources: 1) there are stylet/needle tip morphologies that are more damaging to tissue, and 2) unintended movement of the trocar/needle during the procedure can lacerate the kidney. In certain embodiments, of the stylet points featured in FIGS. 18A-D, conical blunt is recommended for piercing the kidney and minimizing trauma. In certain embodiments, the needle tip is blunt, and non-cutting. Needle gauge size can also add to trauma. In certain embodiments, smaller needles cause less trauma, yet can be ineffective at piercing a fibrotic capsule. In certain embodiments, large needle sizes are more readily viewed by ultrasound for placement considerations. Thus, in certain embodiments, in a design solution that includes a dynamic mode to pierce the capsule effectively with a small needle, there would be less trauma and less risk of failing to penetrate the capsule, but potential visualization problems. In certain embodiments, large differences between sleeve and needle could lead to bending of the small needle within the sleeve and the device is configures to minimize bending of the needle within the sleeve.

### Injection Needle

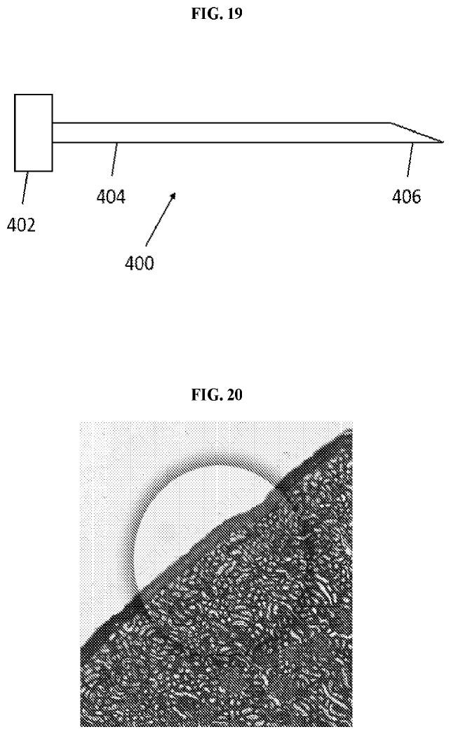

FIG. 19 illustrates a non-limiting example of an injection needle 400 that is configured to engage with the device 100 and be inserted through the trocar 200 when the trocar 200 is in place in a patient. In certain embodiments, the needle 400 is configured to deliver pharmaceutical fluid formulation (such as formulations comprising cells and/or a product thereof) to a target internal tissue site, such as an organ (e.g., a kidney), from the device 100. The illustrated needle 400 has an engagement head 402 and an elongate shaft 404 with a lumen therethrough and a distal tip 406 thereon. The distal tip 406 has a hole therein to allow fluid to flow distally from the needle 400. The engagement head 402 is configured to be removably and replaceably attached to the device 100 along the axis LI at the engagement mechanism 110. When the head 402 is attached, it is configured to engage with the valve 120. Upon actuation of the device 100, the head 402 and the lumen of the elongate shaft 404 are configured to create a fluid flow path for fluid in the fluid reservoir connected to the fluid receiver 106 from the fluid reservoir, through the valve 120, along the lumen of the shaft 404, and out an opening in the distal tip 406. The needle 400 is also configured to be retracted from its position in the target tissue at a as fluid is delivered upon actuation. The size, length, and gauge of the injection needle 400 can vary. For example, the needle 400 can be from about 25 g to about 20 g. Needle gage size can add to trauma of the tissue site, and larger gage sizes (which are smaller needles) can cause less trauma.

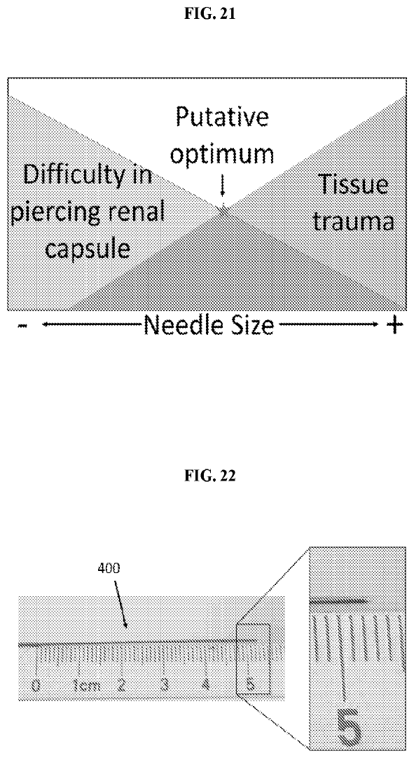

When considering needle size as applied to treating an organ (e.g., a kidney), a balance can be sought between renal capsule penetration and potential trauma to the tissue. As illustrated in FIG. 20, the renal capsule is a tough fibrous layer that surrounds the kidney. Thus a larger needle is more useful for piercing the renal capsule. However, a larger needle can cause more trauma to the kidney. An optimal needle size can consequently be sought that takes these contrary factors into consideration, as illustrated in FIG. 21.

While smaller needles can be less effective at piercing tissue, the needle 400 used in conjunction with the trocar 200 and the stylet 300 here can be configured to allow both successful piercing of tissue and placement of a smaller needle. A variety of different needles can be used herein. Table 2 illustrates additional exemplary needle gage sizes that can optionally be used herein.

TABLE 2

| Needle | Nominal | | OD Range | Nominal | | | Wall |
|---|---|---|---|---|---|---|---|
| Gauge | OD (in) | OD (mm) | (in) | ID (in) | ID (mm) | ID Range (in) | (in) |
| 20 | 0.036 | 0.91 | 0.0355-0.0360 | 0.024 | 0.6 | 0.0230-0.0245 | 0.006 |
| 20.5 | 0.0343 | 0.87 | 0.0340-0.0345 | 0.0265 | 0.67 | 0.0255-0.0275 | 0.004 |
| 21 | 0.032 | 0.82 | 0.0320-0.0325 | 0.02 | 0.51 | 0.0195-0.0210 | 0.006 |
| 21.5 | 0.0303 | 0.77 | 0.0300-0.0305 | 0.0238 | 0.6 | 0.0230-0.0245 | 0.0035 |
| 22 | 0.028 | 0.72 | 0.0280-0.0285 | 0.016 | 0.41 | 0.0155-0.0170 | 0.006 |
| 22 s | 0.028 | 0.718 | 0.0280-0.0285 | 0.007 | 0.168 | 0.0055-0.0077 | 0.011 |
| 22.5 | 0.0263 | 0.67 | 0.0260-0.0265 | 0.0203 | 0.52 | 0.0195-0.0210 | 0.003 |
| 23 | 0.025 | 0.64 | 0.0250-0.0255 | 0.013 | 0.34 | 0.0125-0.0140 | 0.006 |
| 23 s | 0.025 | 0.642 | 0.0250-0.0255 | 0.005 | 0.116 | 0.0040-0.0051 | 0.01 |
| 23.5 | 0.0233 | 0.59 | 0.0230-0.0235 | 0.0173 | 0.44 | 0.0165-0.0180 | 0.003 |
| 24 | 0.022 | 0.57 | 0.0220-0.0225 | 0.012 | 0.31 | 0.0115-0.0130 | 0.005 |
| 24.5 | 0.0213 | 0.54 | 0.0210-0.0215 | 0.0163 | 0.41 | 0.0155-0.0170 | 0.002 |
| 25 | 0.02 | 0.51 | 0.0200-0.0205 | 0.01 | 0.26 | 0.0095-0.0110 | 0.005 |
| 25 s | 0.02 | 0.515 | 0.0200-0.0205 | 0.006 | 0.153 | 0.0055-0.0065 | 0.007 |

In certain embodiments, the needle is a 18 to 30 gauge needle. In certain embodiments, the needle is smaller than 20 gauge. In certain embodiments, the needle is smaller than 21 gauge. In certain embodiments, the needle is smaller than 22 gauge. In certain embodiments, the needle is smaller than 23 gauge. In certain embodiments, the needle is smaller than 24 gauge. In certain embodiments, the needle is smaller than 25 gauge. In certain embodiments, the needle is smaller than 26 gauge. In certain embodiments, the needle is smaller than 27 gauge. In certain embodiments, the needle is smaller than 28 gauge. In certain embodiments, the needle is smaller than 29 gauge. In certain embodiments, the needle is about 20 gauge. In certain embodiments, the needle is smaller than 29 gauge. In certain embodiments, the needle is about 21 gauge. In certain embodiments, the needle is smaller than 29 gauge. In certain embodiments, the needle is about 22 gauge. In certain embodiments, the needle is smaller than 29 gauge. In certain embodiments, the needle is about 23 gauge. In certain embodiments, the needle is smaller than 29 gauge. In certain embodiments, the needle is about 24 gauge. In certain embodiments, the needle is smaller than 29 gauge. In certain embodiments, the needle is about 25 gauge. In certain embodiments, the needle is smaller than 29 gauge. In certain embodiments, the needle is about 26 gauge. In certain embodiments, the needle is smaller than 29 gauge. In certain embodiments, the needle is about 27 gauge. In certain embodiments, the needle is smaller than 29 gauge. In certain embodiments, the needle is about 28 gauge. In certain embodiments, the needle is smaller than 29 gauge. In certain embodiments, the needle is about 29 gauge.

In certain embodiments, the inner diameter of the needle is less than 0.84 mm. In certain embodiments, the inner diameter of the needle is less than 0.61 mm. In certain embodiments, the inner diameter of the needle is less than 0.51 mm. In certain embodiments, the inner diameter of the needle is less than 0.41 mm. In certain embodiments, the inner diameter of the needle is less than 0.33 mm. In certain embodiments, the inner diameter of the needle is less than 0.25 mm. In certain embodiments, the inner diameter of the needle is less than 0.20 mm. In certain embodiments, the inner diameter of the needle is less than 0.15 mm. In certain embodiments, the outer diameter of the needle is less than 1.27 mm. In certain embodiments, the outer diameter of the needle is less than 0.91 mm. In certain embodiments, the outer diameter of the needle is less than 0.81 mm. In certain embodiments, the outer diameter of the needle is less than 0.71 mm. In certain embodiments, the outer diameter of the needle is less than 0.64 mm. In certain embodiments, the outer diameter of the needle is less than 0.51 mm. In certain embodiments, the outer diameter of the needle is less than 0.41 mm. In certain embodiments, the outer diameter of the needle is less than 0.30 mm. In certain embodiments, a needle has one of the sizes in the following table:

| | |
|---|---|
| ID Size | OD Size |

| Gauge | in | mm | in | mm |
|---|---|---|---|---|
| 14 | 0.060 | 1.55 | 0.072 | 1.83 |
| 15 | 0.054 | 1.37 | 0.065 | 1.65 |
| 16 | 0.047 | 1.19 | n/a | n/a |
| 18 | 0.033 | 0.84 | 0.050 | 1.27 |
| 20 | 0.023 | 0.61 | 0.036 | 0.91 |
| 21 | 0.020 | 0.51 | 0.032 | 0.81 |
| 22 | 0.016 | 0.41 | 0.028 | 0.71 |
| 23 | 0.013 | 0.33 | 0.025 | 0.64 |
| 25 | 0.010 | 0.25 | 0.020 | 0.51 |
| 27 | 0.008 | 0.20 | 0.016 | 0.41 |
| 30 | 0.006 | 0.15 | 0.012 | 0.30 |
| 32 | 0.004 | 0.10 | 0.009 | 0.23 |

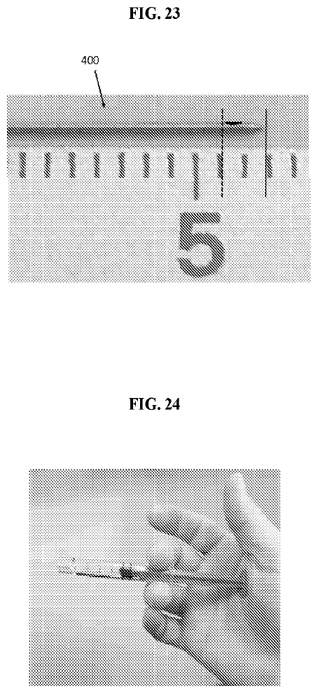

The needle 400 can, for example, inject at an internal tissue site that is a relatively long distance away from the device 100, such as from about 10 cm to 20 cm away. In such an example, the trocar 200 can also mechanically support the needle 400 against kinking that may develop as a result of penetration into the internal tissue site at a relatively large distance. In certain embodiments, a length of the needle 400 can be configured such that from about 5 cm to about 6 cm of a distal part of the needle 400 extends beyond a distal-most end of the trocar 200. For example, about 5.3 cm (e.g., about 5.5, 6, or 6.5 cm) of the needle 400 can be configured to extend distally beyond the distal-most end of the trocar 200, as illustrated in FIG. 22. The length can allow sufficient penetration depth of the needle 400 within a target tissue, such as an organ (e.g., a kidney), for deposition of the therapeutic cells or cell products. In certain embodiments of the injection device, penetration depths of the needle can be variably set. In certain embodiments, at such a length, the hole in the distal tip 406 of the needle 400 can be positioned about 5.1 cm past the distal-most end of the trocar 200 as illustrated in FIG. 23. In certain embodiments, the needle hole is preferentially laterally-located about 0.2 cm from the tip of the needle, and the hole is located at 5.1 cm past the trocar on a 5.3 cm length. In certain embodiments, relating to an about 6 cm extension, the hole is located at about 5.8 cm past the trocar. In certain embodiments, the lateral positioning of the exit hole has a beneficial effect on extrusion and placement of the cell therapeutic while ensuring the needle is non-coring. In certain embodiments, the hole can optionally be laterally located, which can provide a beneficial effect on extrusion and placement of the therapeutic cells or cell products in the target tissue site. In various examples, one or more surface features can also be added to an outer distal surface of the elongate shaft 404 of the needle 400) similar to the trocar 200, such as bumps, grooves, markings, etc., that are configured to provide better visualization of a location of the needle 400, such as under ultrasound, and thus more accurate placement within a patient. More than one needle 400 of varying sizes can also be used depending on the desired treatment. In some examples, needle gage size can add to trauma. For instance, larger gage sizes (which are smaller needles) can cause less trauma, yet can be ineffective at piercing tissue, such as a fibrotic capsule. Therefore, provided herein are components, such as the trocar 200, that can assist through a dynamic mood and/or dynamic movement to pierce tissue, such as the capsule, effectively with a small needle. This can result in less trauma and less risk of failing to penetrate the tissue, such as the capsule.

In certain embodiments, the components herein can be designed in a fashion that accommodates needs of a user, such as a surgeon utilizing the device. In use, all of the components herein, such as moving surfaces, can preferably be comfortable and fit in a wide range of male and/or female hand sizes. Anti-slip surfaces can optionally be integrated into one or more components discussed herein, for example providing surface features, surfaces, and/or materials that interact well with and prevent slippage in surgical gloves. In certain embodiments, input interfaces can be easily seen and easily activated with gloved fingers. In certain embodiments, one or more of the interfaces can provide tactile feedback when activated, and if any part of the injection process is mechanical, tactile feedback can be important to successful use of the components disclosed herein, for example as a fluid such as a therapeutic cellular material is injected into tissue such as a renal space. Users often express a preference for smaller syringes because the smaller syringes require less force for infusing contents thereof, and smaller syringes are sometimes held in atypical hand positions for better control, as illustrated in a non-limiting example in FIG. 24. Thus ergonomics and comfort of fit of the components herein can be taken into consideration. In certain embodiments, one or more instructions, instructions for use, guides, videos, operator's manuals, etc. can be provided, and they can be tailored to needs of an end user (such as a surgeon). The instructions, etc. can be configured to be in accordance with and/or defined by the needs of the Contract Engineering Organization's design process.

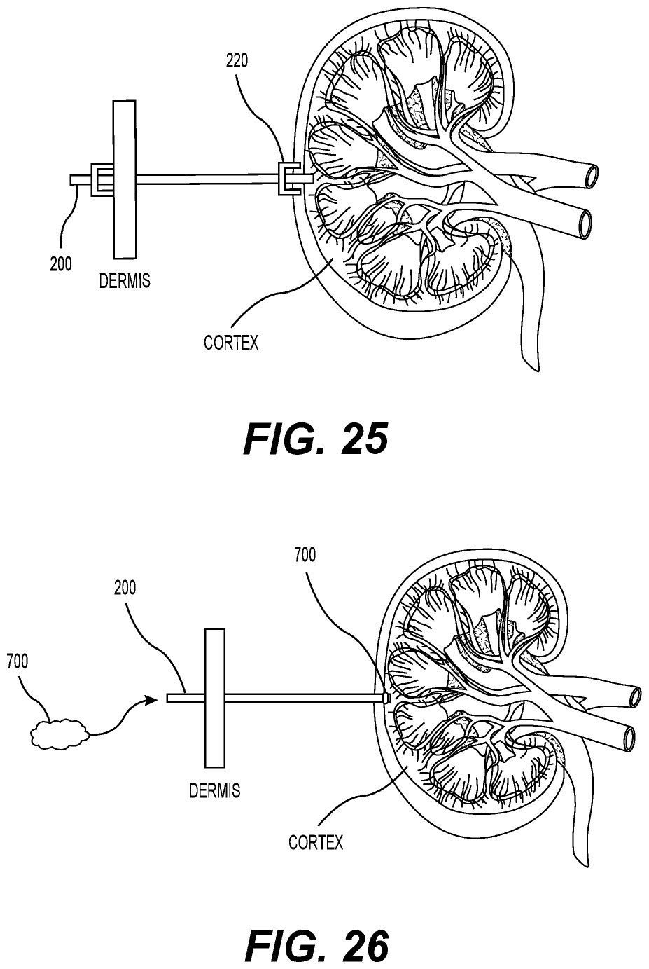

The device 100, trocar 200, and needle 400 can be used in a variety of different ways. For example, the injection device 100 can be attached to the trocar 200 with the stylet 300 in place within the trocar 200. The cartridge 130 or other fluid reservoir can be engaged with the device 100 at this point or prior to injection. The trocar 200 and stylet 300 can then be maneuvered to penetrate an outer tissue surface, such as the dermis, and an inner tissue target site, such as an organ (e.g., a kidney), of a patient at a desired depth (as discussed above). The stylet 300 can then be removed from the trocar 200 through the removal lumen 140. Removal of the stylet 300 can trigger actuation of the stabilization mechanism 220 on the trocar 200. As illustrated in FIG. 12, the feet 222 can deploy to grasp or engage tissue. The trocar 200 can be disconnected from the device 100, and at this point during use, the trocar 200 will be supported through frictional interaction with the dermis and through employment of the stabilization mechanism 220. In a non-limiting illustrative example, FIG. 25 illustrates a simplified diagram of the trocar 200 in use on a kidney, in which the trocar 200 is experiencing frictional interaction with the dermis and is experiencing stabilization through employment of the stabilization mechanism 220 against a cortex of the kidney, which is an outer portion of the kidney between the renal capsule and the renal medulla that can vary in thickness from patient to patient. For example, cortical thickness can be from about 3 to 12 mm, and more particularly from about 3.2 to 11 mm with a mean value of about 5.9 mm. The injection needle 400 can be engaged with the device 100. The needle 400 can then be inserted into the trocar 200 until the needle 400 penetrates the target tissue through the same hole created by the stylet 300 at a desired depth (as discussed above), and the actuator 104 on the device 100 can be actuated to deliver fluid, such as therapeutic cells or cell products, to the target site. The stabilization mechanism 220 can work in conjunction with the compressive spring section 208 of the trocar 200 and the flexible mounting of the valve 120 to absorb motion of the tissue site, such as an organ (e.g., a kidney), relative to the user's hold on and actuation of the device 100. As the fluid is delivered, the needle 400 can be retracted from its penetrating position within the target tissue site. The trigger lever activates an electromechanical system that delivers pulses of therapeutic solution within the needle tract as the needle is retracted. Multiple injections to the same tissue site, such as an organ, can be performed.

Alternatively, the trocar 200 and the stylet 300 can be maneuvered into place within the patient without being attached to the device 100, and the stylet 300 can be removed to actuate the stabilization mechanism 220 on the trocar 200. The needle 400 with the device 100 engaged can then be inserted into the trocar 200 for delivery of fluid. The trocar 200 with the stylet 300 can also be positioned within a patient while attached to the device 100, and then the device 100 can be detached from the trocar 200 to manually remove the stylet 300 before attaching the needle 400 and inserting into the trocar 200. If the trocar 200 has an additional engagement mechanism, such as the reversibly collapsible, expanding element 500, the injection device 100 can be attached to the trocar 200 with the stylet 300 in place within the trocar 200. The trocar 200 and stylet 300 can then be maneuvered to penetrate the outer tissue surface and the inner tissue target site. After removing the stylet 300 and disconnecting the device 100 but prior to insertion of the needle 400, the expanding element 500 can be deployed. However, this order can change depending on the employment mechanism of different engagement mechanisms. When the device 100 is used specifically to treat a kidney, the trocar 200, stylet 300, and needle 400 can be used as discussed above to pierce the capsule of the kidney, a region of potentially fibrotic tissue, and deliver a proscribed volume to the kidney while simultaneously withdrawing the needle 400 to allow expansion space for the delivered bolus of therapeutic cells or cell products. As used herein and depending on context, “fibrotic” means or denotes deposition of fibrous extracellular matrix, which is usually denser than surrounding tissue. Densely fibrotic tissue is often a result of a chronic disease state. In certain embodiments, fibrotic tissue is a result of a chronic disease state. In certain embodiments, densely fibrotic tissue is a result of a chronic disease state. In certain embodiments, fibrotic tissue is present in an organ undergoing treatment with (e.g., is the delivery site of a composition delivered using) a device, system, or method provided herein.

In certain embodiments, prior to use the cartridge 130 can be warmed to about 25 to 30° C., and more particularly to about 26 to 28° C. In certain embodiments, prior to use the cartridge 130 can be warmed to about 25, 26, 27, 28, 29, 30, 31, 32, 33, 34, 35, 36, or 37° C. In certain embodiments, warming can occur over a controlled period of time, such as over about 5, 10, 15, 20, 25, 30, 35, 40, 45, 50, 55, or 60 minutes, or about 15 to about 45 minutes. In certain embodiments, once the cartridge 130 has been warmed, delivery of the fluid therein can preferably occur within about 0.5, 1, 1.5, 2, or 2.5 hours to avoid discarding the cartridge 130.

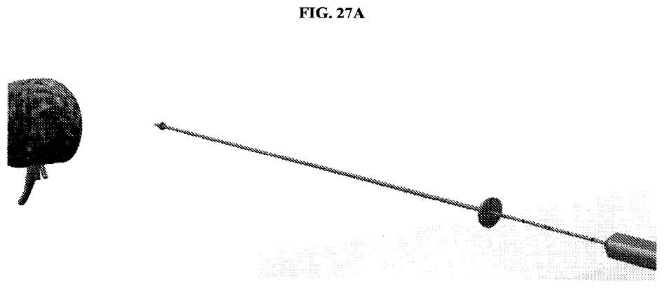

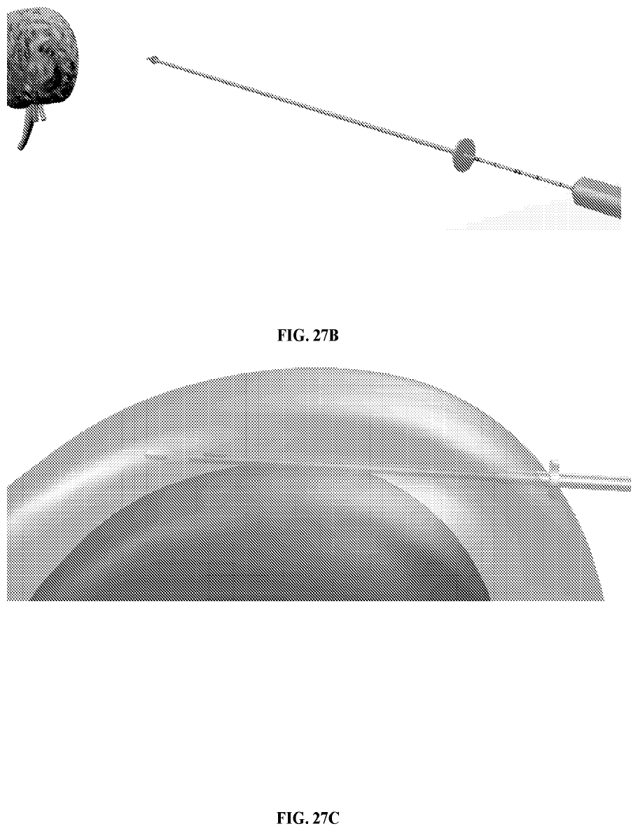

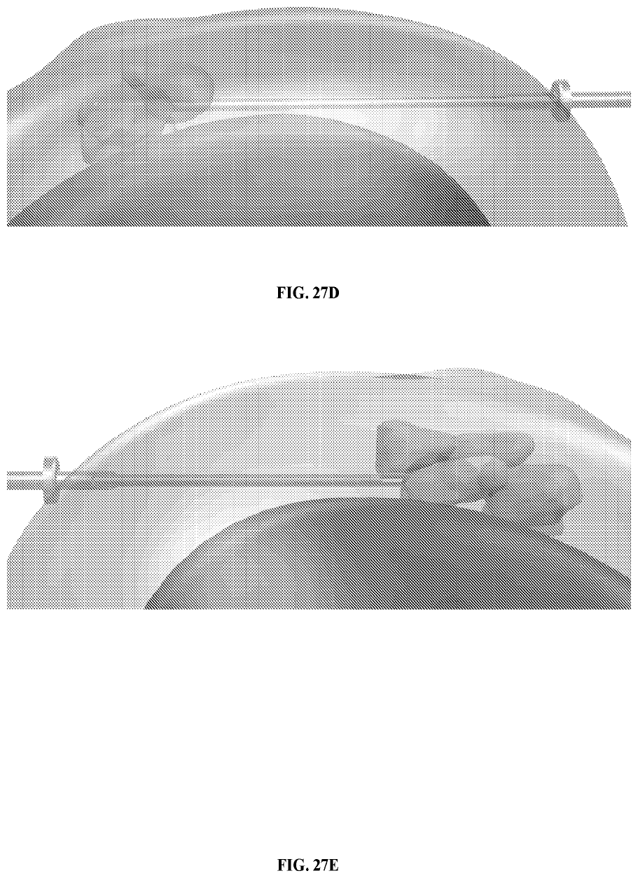

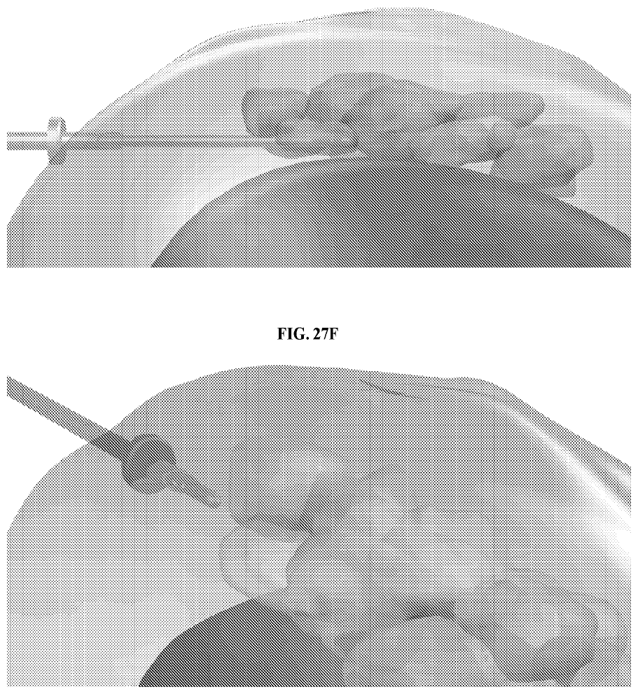

In certain embodiments, after delivery of the fluid and removal of the injection needle 400 from the target tissue site and the trocar 200, a plug 700 (such as a hydrogel plug or pledget, such as a Gelfoam® pledget) can be manually inserted into the trocar 200 and pushed down and out of the distal end 206 of the trocar 200 to seal the penetration wound. The device 100 and the trocar 200 can be configured to accommodate delivery of the plug. In certain embodiments, a pledget (such as a Gelfoam® pledget) that starts out as about 2 to about 4 mm by about 10 mm is compressed to allow insertion (e.g., manual insertion) into the trocar and pushed down and out of the tip of the trocar to seal the wound at the kidney capsule. In certain embodiments, the pledget is a small wad of absorbent cotton or other soft material (such as a hydrogel). In certain embodiments, the pledget or plug is used to stop up a wound or other opening in a body or organ. In certain embodiments, the device is configured to accommodate this terminal wound sealing process. In a non-limiting illustrative example, FIG. 26 illustrates a simplified diagram of the trocar 200 in use on a kidney in which the plug 700 is being “musket loaded” or manually put into place through the trocar 200. The trocar 200 can then be removed from the patient. Thus throughout the process disclosed herein, one or more of the components can assist in manual piercing of tissue, such as fibrotic kidney capsules, smooth, controlled delivery of fluid, such as the therapeutic cellular material, and positional stability of the trocar in the dynamic environment of the patient (which in turn provides stability to delivery of the therapeutic cellular material while minimizing trauma to tissue), which have presented challenges in other approaches but one or more current components herein can be configured to address.

While focusing on a kidney as the target tissue site and/or organ has been described, components herein can have broad application for numerous organs and/or internal tissue sites and/or respective therapies. As a non-limiting example, components discussed herein are thus hand-held injection components designed to deliver one or more injection volumes (such as cell therapy) to one or more target tissue sites, such as the parenchymal and/or stromal compartments of a diseased site like an organ (such as the kidney or tumor site). In certain embodiments, the components can thus pierce an outer surface of the tissue site, such as an organ site like the kidney capsule, a region of potentially fibrotic tissue in the kidney, and can deliver a proscribed volume along an injection path while simultaneously withdrawing an injection needle to allow expansion space for a delivered volume, such as a bolus of therapeutic cell material. In certain embodiments, multiple injections to a same target site, such as an organ, can be possible. In certain embodiments, the components herein can have means to stabilize themselves against a surface of a target tissue site, such as an organ, through trocar deployment, can accept pre-filled aseptic cartridges containing a volume, such as the therapeutic cellular material solution, and can be ergonomic to an intended operator, such as a surgeon. In certain embodiments, the component herein can be intuitive and accommodate hypodermic needles within a specified range of sizes and can possess the ability to set variable injection or dispense volumes and penetration depths.

In certain embodiments, the injection device can consist of a single physical device that can accept cartridge-based therapeutic doses. In certain embodiments, standard trocar/sleeve/needle combinations can be incorporated into the device from off-the-shelf, individually packaged and sterilized stores. In such examples, targeted sizes can be swapped out from various standard sizes, such as hypodermic needles being in the range of about 25 g minimum to about 20 g maximum and trocar/sleeve combinations of at least about 20 g. In certain embodiments, the trocar/sleeve size is from about 21 g to about 18 g. In certain embodiments, a subject has a high body mass index (e.g., a BMI of at least about 35, 36, 37, 38, 39, or 40 kg/m²). BMI is a measure of obesity, and a high BMI is associated with high obesity. In certain embodiments, sleeve size of about 20 g to about 18 g and a needle of about 21 g and a length of about 20 to about 30 (e.g., 25 cm) is used for patients with a high BMI. In certain embodiments, provided herein is a device or system that is useful for treating both normal and high BMI patients. In certain embodiments, an injection path of the injection needle to a target site, such as an organ, can be provided by pairing with a trocar. In certain embodiments, the trocar can serve to mechanically support the injection needle against kinking that can develop in some instances as a result of penetration into the target tissue, such as the organ, at a relatively large distance, such as about 5 to 25 cm and more particularly about 10 to 20 cm, away from the injection device.

In certain embodiments of one or more of the components herein, components can be labeled with ISO compliant safety labels in accordance with appropriate regulatory agency documentation (e.g. United States 21 C.F.R. § 801). In certain embodiments, the components herein can be considered safe for uncontrolled access when various housing and product skins are in place and secured, and components herein can optionally have no externally accessible sharp edges with a radius of less than about 0.5 mm excluding the various trocar, sleeve, and needle components. In certain embodiments, the components can have no externally accessible electrical connection that is capable of sourcing greater than about 1.0 A at about 5.25 VDC. In certain embodiments, any materials, such as plastics, and/or finishes can be flammability rated UL94 V-0 or better.

In certain embodiments, the components herein can be configured to be used in temperature-controlled indoor environments, such as clinics and hospitals, and the components can be configured to meet performance requirements over a range of environmental conditions as provided in Table 3:

TABLE 3

| Parameter (units) | Min | Max |
|---|---|---|
| Temperature (° C.) | 28 | 37 |
| Relative humidity (%) | 20 | 90 |
| Ambient pressure (bar) | 0.7 | 1.05 |
| Light level (lux) | 100 | 1000 |
| Vibration (g RMS) | 0 | 0.1 |

In certain embodiments, the components disclosed herein can be configured to operate normally after various sterilization processes (e.g., standard sterilization processes), such as through gamma radiation, ethylene oxide, e-beam, or gas. In certain embodiments incorporating one or more plastics, the plastics can be configured to not become embrittled by the sterilization process, and decolorization or color change can be minimal. In certain embodiments herein that incorporate one or more electromechanical components, sterilization processes can be used that are compatible with electronics, which can incorporate various steps and additional features, such as removing removable skins and/or surfaces that can be sterilized and aseptically contain working elements of one or more of the components. Table 4 and Table 5 provide resin materials that are compatible with various types of sterilization.

TABLE 4
Plastics Compatibility with Sterilization Methods

| | | | | | | |
|---|---|---|---|---|---|---|
| | | | | Ethylene | | |
| | Polymer | | Dry | Oxide | Gamma | Electron |
| Polymer | Abbreviation | Autoclave | Heat | (Eto) | Irradiation | Beam |

Biopolymers

| | | | | | | |
|---|---|---|---|---|---|---|
| Poly(L-lactide) | PLLA | Fair | Good | Good | Good | Good |
| Polyactic acid | PLA | Poor | Fair | Good | Good | Good |
| Polyhydroxybutyrate | PHB | Poor | Poor | Good | Fair | Fair |
| Polyglycolic acid | PGA | Good | Good | Good | Good | Good |
| Poly(lactic-co-glycolic acid) | PLGA | Poor | Poor | Good | Fair | Fair |
| Porycaprolactone | PCL | Fair | Good | Good | Good | Good |

Elastomers

| | | | | | | |
|---|---|---|---|---|---|---|
| Silcones | VMQ, PMQ, | Good | Good | Good | Good | Good |
| | PVMQ | | | | | |
| Urethane thermoplastic | TPU | Poor | Fair | Good | Good | Good |
| elastomer | | | | | | |
| Copolyester thermoplastic | TPC | Poor | Good | Good | Good | Good |
| elastomer | | | | | | |
| Polyamide thermoplastic | TPA | Poor | Poor | Good | Good | Good |
| elastomer | | | | | | |
| Styrenic thermoplastic | TPS | Poor | Poor | Good | Good | Good |
| elastomer | | | | | | |
| Olefinic thermoplastic | TPO | Poor | Fair | Good | Good | Good |
| elastomer | | | | | | |

Fluoropolymers

| | | | | | | |
|---|---|---|---|---|---|---|
| Polytetrafluoroethylene¹ | PTFE | Fair | Fair | Good | Poor | Poor |
| Fluorinated ethylene | FEP | Good | Good | Good | Fair | Fair |
| propylene | | | | | | |
| Perfluoro alkoxy | PFA | Good | Good | Good | Good | Good |
| Ethylene | ECTFE | Good | Good | Good | Good | Good |
| chlorotrifluoroethylene | | | | | | |
| Ethylene | ETFE | Good | Good | Good | Good | Good |
| tetrafluoroethylene | | | | | | |
| Polyvinyl fluoride | PVF | Good | Good | Good | Good | Good |
| Polyvinylidene difluoride | PVF2 | Good | Good | Good | Good | Good |

High-temperature thermoplastics

| | | | | | | |
|---|---|---|---|---|---|---|
| Porysulfones | PSU | Good | Good | Good | Good | Good |
| Polyphenylene sulfide | PPS | Good | Good | Good | Good | Good |
| Liquid crystalline polymer | LCP | Good | Good | Good | Good | Good |
| Polyetherimide | PEI | Fair | Fair | Good | Good | Good |
| Polyamide-imide | PAI | Fair | Fair | Good | Good | Good |
| Polyetheretherketone | PEEK | Good | Good | Good | Good | Good |

¹Radiation stable grades need to be used for radiation sterilization.

TABLE 5
Plastics Compatibility with Sterilization Methods

| | | | | Ethylene | | |
|---|---|---|---|---|---|---|
| | Polymer | | Dry | Oxide | Gamma | Electron |
| Polymer | Abbreviation | Autoclave | Heat | (Eto) | Irradiation | Beam |
| Nylon 6, Nylon 66 | PA6, PA66 | Fair | Fair | Good | Fair | Fair |
| Aromatic | AP | Good | Good | Good | Good | Good |
| Nylon 12, 6/12 | PA12 | Poor | Poor | Good | Fair | Fair |

Polyesters

| | | | | | | |
|---|---|---|---|---|---|---|
| Poly butylene terephthalate | PBT | Fair | Fair | Good | Good | Good |
| Poly ethylene terephthalate | PET | Poor | Poor | Good | Good | Good |
| Copolyesters | | Poor | Poor | Good | Good | Good |

Polyolefins

| | | | | | | |
|---|---|---|---|---|---|---|
| High-density polyethylene | HOPE | Poor | Poor | Good | Good | Good |
| Low-density polyethylene | LDPE | Poor | Poor | Good | Good | Good |
| Ultrahigh molecular weight | UHMWPE | Poor | Poor | Good | Good | Good |
| polyethylene | | | | | | |
| Polypropylene¹ | PP | Good | Fair | Good | Far | Fair |
| Polypropylene copolymers | PPC | Good | Fair | Good | Fair | Fair |
| Cyclo olefin copolymer | COC | Fair | Fair | Good | Good | Good |
| Polyvinyl chloride | PVC | Fair | Fair | Good | Good | Good |
| plasticized^{1,2} | | | | | | |
| Polyvinyl chloride | PVC | Poor | Poor | Good | Fair | Fair |
| unplasticized^{1,2} | | | | | | |

Polystyrene/Styrenics

| | | | | | | |
|---|---|---|---|---|---|---|
| Polystyrene | PS | Poor | Poor | Good | Good | Good |
| Acrylonitrile butadiene | ABS | Poor | Poor | Good | Good | Good |
| styrene copolymer (Abs) | | | | | | |
| Styrene-acrylonitrile | SAN | Poor | Poor | Good | Good | Good |
| copolymer (San) | | | | | | |
| Acrylonitrile styrene | ASA | Poor | Poor | Good | Good | Good |
| acrylate | | | | | | |
| Methacrylate acrylonitrile | MABS | Poor | Poor | Good | Good | Good |
| butadiene styrene | | | | | | |
| copolymer | | | | | | |
| Styrene-butadiene | SBC | Poor | Poor | Good | Good | Good |
| copolymer | | | | | | |
| Acrylics^{1,2} | PMMA | Poor | Poor | Good | Good | Good |
| Polycarbonates^{1,2} | PC | Fair | Fair | Good | Good | Good |
| High heat polycarbonates | PC | Good | Good | Good | Good | Good |
| Polyurethanes | PU, PUR | Poor | Poor | Good | Good | Good |
| Acetals | POM | Good | Good | Good | Poor | Poor |

Polyamides
¹Radiation stable grades need to be used for radiation sterilization
²PVC, acrylics, and PC require corrective tint to compensate for discoloration.

In certain embodiments, one or more components herein that have been packaged can be configured to operate normally when returned to operating environmental range after an extended period of time. For example, Table 6 illustrates packaging being exposed to various ranges of environmental conditions over 72 hours of storage, after which the components operated normally:

TABLE 6

| Parameter | Min | Max |
|---|---|---|
| Temperature (° C. 0 | 0 | 50 |
| Relative humidity (%) | 20 | 90 |
| Ambient pressure (bar) | 0.7 | 1.05 |

In certain embodiments that have been packaged, packaged components can be configured and packaged such that they do not suffer functional or visible cosmetic damage when shipped by commercial carriers. Additionally, various packaged embodiments of components herein can be configured and packaged such that they do not suffer functional or visible cosmetic damage when dropped from a height of about 1 meter.

Each component discussed above can be used independently of each other, used entirely together, or any combination of the two. For example, components can be provided independently or can be provided in various combinations, systems, and kits to end users. Various combinations of cartridges 130 and/or size ranges and types of needles 400, trocars 200, and/or stylets 300 can also be provided with the device 100. In certain embodiments, one or more components or all components together can be packaged and shipped as a fully assembled unit ready to operate by the end-user upon removal from the package. In certain embodiments, instructions for assembly and/or use can be provided with any components, for example allowing an end-user to assemble or configure one or more for operation in less than about 10 minutes.

### Non-Limiting Examples of Injectable Formulations

The devices and systems provided herein may be configured for the delivery of different pharmaceutical fluid formulations. In certain embodiments, the fluid formulations comprise an active agent (such as a cell, cell product, or compound) and a temperature-sensitive biomaterial. In certain embodiments, the temperature-sensitive biomaterial is a pharmaceutically acceptable carrier for the active agent. In certain embodiments, a formulation incorporates biomaterials having properties that create a favorable environment for the active agent, such as bioactive renal cells, to be administered to a subject.

In certain embodiments, fluid formulation is capable of becoming a hydrogel, e.g., the formulation is a hydrogel that is above a melting temperature.

In certain embodiments, devices and systems provided herein are configured to deliver a formulation (such as NKA) that has been heated to a temperature sufficient to melt or otherwise ensure that the composition is a liquid. In certain embodiments, the device is configured to warm a formulation or maintain a temperature at which the formulation is a liquid.

In certain embodiments, temperature sensitivity of the formulation can be varied by adjusting the percentage of a biomaterial in the formulation. For example, the percentage of gelatin in a solution can be adjusted to modulate the temperature sensitivity of the gelatin in the final formulation (e.g., liquid, gel, beads, etc.).

In certain embodiments, the temperature-sensitive biomaterial may have (i) a substantially solid state at about 8° C. or below, and (ii) a substantially liquid state at ambient temperature or above. In certain embodiments, the ambient temperature is about room temperature.

In certain embodiments, the state of the temperature-sensitive biomaterial is a substantially solid state at a temperature of about 8° C. or below. In certain embodiments, the substantially solid state is maintained at about 1° C., about 2° C., about 3° C., about 4° C., about 5° C., about 6° C., about 7° C., or about 8° C. In certain embodiments, the substantially solid state has the form of a gel. In certain embodiments, the state of the temperature-sensitive biomaterial is a substantially liquid state at ambient temperature or above. In certain embodiments, the substantially liquid state is maintained at about 25° C., about 25.5° C., about 26° ° C., about 26.5° ° C., about 27° C., about 27.5° C., about 28° C., about 28.5° C., about 29° ° C., about 29.5° C., about 30° C., about 31° C., about 32° C., about 33° C., about 34° C., about 35° C., about 36° C., or about 37° C. In certain embodiments, the ambient temperature is about room temperature.

In certain embodiments, the state of the temperature-sensitive biomaterial is a substantially solid state at a temperature of about ambient temperature or below. In certain embodiments, the ambient temperature is about room temperature. In certain embodiments, the substantially solid state is maintained at about 17° C., about 16° ° C., about 15° C., about 14° C., about 13° C., about 12° C., about 11° C., about 10° C., about 9° C., about 8° C., about 7° C., about 6° C., about 5° C., about 4° C., about 3° C., about 2° C., or about 1° C.

In certain embodiments, cell populations and preparations to be delivered may be coated with, deposited on, embedded in, attached to, seeded, suspended in, or entrapped in a temperature-sensitive biomaterial. In certain embodiments, the cell populations may be assembled as three dimensional cellular aggregrates or spheroids or three dimensional tubular structures in the temperature-sensitive biomaterial.

In certain embodiments, the temperature-sensitive biomaterial has a transitional state between a first state and a second state. In certain embodiments, the transitional state is a solid-to-liquid transitional state between a temperature of about 8° C. and about ambient temperature. In certain embodiments, the ambient temperature is about room temperature. In certain embodiments, the solid-to-liquid transitional state occurs at one or more temperatures of about 8° C., about 9° C., about 10° C., about 11° C., about 12° C., about 13° C., about 14° C., about 15° C., about 16° C., about 17° C., and about 18° C.

In certain embodiments, the temperature-sensitive biomaterials have a certain viscosity at a given temperature measured in centipoise (cP). In certain embodiments, the biomaterial has a viscosity at 25° C. of about 1 cP to about 5 cP, about 1.1 cP to about 4.5 cP, about 1.2 cP to about 4 cP, about 1.3 cP to about 3.5 cP, about 1.4 cP to about 3.5 cP, about 1.5 cP to about 3 cP, about 1.55 cP to about 2.5 cP, or about 1.6 cP to about 2 cP. In certain embodiments, the biomaterial has a viscosity at 37° C. of about 1.0 cP to about 1.15 cP. The viscosity at 37° C. may be about 1.0 cP, about 1.01 cP, about 1.02 cP, about 1.03 cP, about 1.04 cP, about 1.05 cP, about 1.06 cP, about 1.07 cP, about 1.08 cP, about 1.09 cP, about 1.10 cP, about 1.11 cP, about 1.12 cP, about 1.13 cP, about 1.14 cP, or about 1.15 cP. In certain embodiments, the biomaterial is a gelatin solution. The gelatin is present at about 0.5%, about 0.55%, about 0.6%, about 0.65%, about 0.7%, about 0.75%, about 0.8%, about 0.85%, about 0.9%, about 0.95% or about 1%, (w/v) in the solution. In one example, the biomaterial is a 0.75% (w/v) gelatin solution in PBS. In certain embodiments, the 0.75% (w/v) solution has a viscosity at 25° ° C. of about 1.6 cP to about 2 cP. In certain embodiments, the 0.75% (w/v) solution has a viscosity at 37° ° C. of about 1.07 cP to about 1.08 cP. The gelatin solution may be provided in PBS, DMEM, or another suitable solvent.

In certain embodiments, a fluid formulation is gelatin-based. Gelatin is a non-toxic, biodegradable and water-soluble protein derived from collagen, which is a major component of mesenchymal tissue extracellular matrix (ECM). Collagen is the main structural protein in the extracellular space in the various connective tissues in animal bodies. As the main component of connective tissue, it is the most abundant protein in mammals, making up from 25% to 35% of the whole-body protein content. Depending upon the degree of mineralization, collagen tissues may be rigid (bone), compliant (tendon), or have a gradient from rigid to compliant (cartilage). Collagen, in the form of elongated fibrils, is mostly found in fibrous tissues such as tendons, ligaments and skin. It is also abundant in corneas, cartilage, bones, blood vessels, the gut, intervertebral discs and the dentin in teeth. In muscle tissue, it serves as a major component of the endomysium. Collagen constitutes one to two percent of muscle tissue, and accounts for 6% of the weight of strong, tendinous muscles. Collagen occurs in many places throughout the body. Over 90% of the collagen in the human body, however, is type I.

To date, 28 types of collagen have been identified and described. They can be divided into several groups according to the structure they form: Fibrillar (Type I, II, III, V, XI). Non-fibrillar FACIT (Fibril Associated Collagens with Interrupted Triple Helices) (Type IX, XII, XIV, XVI, XIX). Short chain (Type VIII, X). Basement membrane (Type IV). Multiplexin (Multiple Triple Helix domains with Interruptions) (Type XV, XVIII). MACIT (Membrane Associated Collagens with Interrupted Triple Helices) (Type XIII, XVII). Other (Type VI, VII). The five most common types are; Type I: skin, tendon, vascular ligature, organs, bone (main component of the organic part of bone). Type II: cartilage (main collagenous component of cartilage) Type III; reticulate (main component of reticular fibers), commonly found alongside type I. Type IV: forms basal lamina, the epithelium-secreted layer of the basement membrane. Type V: cell surfaces, hair and placenta.

Gelatin retains informational signals including an arginine-glycine-aspartic acid (RGD) sequence, which promotes cell adhesion, proliferation and stem cell differentiation. A characteristic property of gelatin is that it exhibits Upper Critical Solution Temperature behavior (UCST). Above a specific temperature threshold, gelatin can be dissolved in water by the formation of flexible, random single coils. Upon cooling, hydrogen bonding and Van der Waals interactions occur, resulting in the formation of triple helices. These collagen-like triple helices act as junction zones and thus trigger the sol-gel transition. Gelatin is widely used in pharmaceutical and medical applications.

In certain embodiments, a fluid injectable cell formulation is based on porcine gelatin, which may be sourced from porcine skin and is commercially available, for example from Nitta Gelatin NA Inc (NC, USA) or Gelita USA Inc. (IA, USA). Gelatin may be dissolved, for example, in Dulbecco's phosphate-buffered saline (DPBS) to form a thermally responsive hydrogel, which can gel and liquefy at different temperatures. In certain embodiments, the hydrogel used to formulate the injectable cell compositions is based on recombinant human or animal gelatin expressed and purified using methodologies known to those of ordinary skill in the art. In certain embodiments, an expression vector containing all or part of the cDNA for Type I, alpha I human collagen is expressed in the yeast Pichia pastoris. Other expression vector systems and organisms will be known to those of ordinary skill in the art. In certain embodiments, a gelatin-based hydrogel of the present disclosure is liquid at and above room temperature (22-28° C.) and gels when cooled to refrigerated temperatures (2-8° C.).

One skilled in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety.

What is claimed is:

1. A pharmaceutical fluid formulation delivery device, comprising:
a body having an actuator, a fluid receiver, and a fluid delivery mechanism; and
a detachable injection needle extending distally from the body,
wherein the fluid delivery mechanism is configured to deliver boluses or a continuous flow of the fluid through the injection needle.

2. The device of claim 1, wherein the fluid delivery mechanism includes an electromechanical system having a central processing unit and a pump.

3. The device of claim 1, further comprising a valve configured to translate proximally and distally parallel to the injection needle during placement of the device and delivery of the fluid.

4. The device of claim 3, wherein the valve is configured to translate about 2 cm distally and proximally.

5. The device of claim 1, wherein the fluid receiver is configured to removably and replaceably receive a fluid reservoir therein, and the fluid reservoir comprises at least one cartridge that includes a known dosage of a fluid.

6. The device of claim 5, wherein the fluid receiver is configured to receive a plurality of cartridges concurrently.

7. The device of claim 1, wherein the fluid includes therapeutic cells or products thereof for the treatment of kidney disease.

8. The device of claim 1, further comprising a touch display configured to control operation of the device.

9. The device of claim 8, wherein the display is configured to set one or more parameters for delivery of the fluid, including at least one of pressure and volume.

10. The device of claim 8, wherein the display is configured to provide real-time dispensing information of the fluid during delivery.

11. The device of claim 1, wherein the actuator is one of a trigger, a plunger, a switch, or a button.

12. The device of claim 1, further comprising an engagement feature on a distal end of the body configured to detachably engage a trocar.

13. A trocar, comprising:
an elongate body having proximal and distal ends, the body having a head on the proximal end thereof, an elongate shaft extending distally from the head, and a lumen extending from the proximal end to the distal end therethrough;
a stabilizing means on a distal portion of the elongate shaft configured to stabilize the distal end of the elongate body relative to a tissue surface.

14. The trocar of claim 13, wherein the stabilizing means includes one or more engagement components that are configured to deploy to releasably grasp the tissue surface upon actuation.

15. The trocar of claim 14, wherein the engagement components include a plurality of feet.

16. The trocar of claim 16, wherein the feet have micro-hooks thereon.

17. The trocar of claim 14, wherein the engagement components include at least one of adhesive, suction, and pinchers.

18. The trocar of claim 14, further comprising a removable stylet configured to extend through the lumen of the elongate body, the stylet being configured to actuate the engagement components upon removal.

19. The trocar of claim 13, wherein at least part of the elongate shaft is configured to translate distally and proximally parallel to a longitudinal axis of the elongate shaft.

20. The trocar of claim 19, wherein the at least part of the elongate shaft is configured to translate about 2 cm distally and proximally.

21. A method of delivering a pharmaceutical fluid formulation to tissue, comprising:
attaching an injection device to a trocar, the trocar having a lumen therethrough and a stylet positioned therein;
connecting a fluid source to the injection device;
advancing the injection device and trocar through an outer tissue surface of a patient and penetrating an inner tissue target site;
removing the stylet from the trocar and disengaging the injection device and the trocar;
attaching an injection needle to the injection device;
inserting the injection needle through the trocar to the tissue target site; and
actuating the injection device to deliver a continuous flow or boluses of a fluid from the fluid source through the injection needle and to the tissue target site.

22. The method of claim 21, further comprising, prior to inserting the injection needle through the trocar, deploying a stabilizing means on a distal portion of the trocar to stabilize the distal end of the trocar relative to the tissue target site.

23. The method of claim 22, wherein deploying the stabilizing means is actuated by removal of the stylet.

24. The method of claim 21, further comprising retracting the injection needle during delivery of the fluid.

25. The method of claim 21, further comprising, during actuating the injection device, stabilizing the injection device using a translating valve and stabilizing the trocar using a compressive spring section of the trocar.
